(19) United States
(12) Patent Application Publication
(10) Pub. No.: US 2011/0295248 A1
(43) Pub. Date: Dec. 1, 2011

(54) SYSTEM AND METHOD FOR AUTOMATED MINIMALLY INVASIVE INSTRUMENT COMMAND

(75) Inventors: Daniel T. Wallace, Santa Cruz, CA (US); Federico Barbagli, San Francisco, CA (US); Alex Goldenberg, San Francisco, CA (US); Randall L. Schlesinger, San Mateo, CA (US); Neal A. Tanner, Mountain View, CA (US)

(73) Assignee: Hansen Medical, Inc., Mountain View, CA (US)

(21) Appl. No.: 12/833,932

(22) Filed: Jul. 9, 2010

Related U.S. Application Data
(60) Provisional application No. 61/349,690, filed on May 28, 2010.

(51) Int. Cl. A61M 25/092; A61B 18/18

(52) U.S. Cl. 606/33; 604/95.01

(57) ABSTRACT

In one embodiment a system may comprise a controller including a master input device; and an electromechanically steerable elongate instrument having proximal interface and portions, the proximal interface portion being configured to be operatively coupled to an electromechanical instrument driver in communication with the controller, the distal portion being configured to be interactively navigated adjacent internal tissue structures of a patient's body in response to signals from the controller; wherein the controller is operatively coupled to a treatment interactivity variable sensor selected from the group consisting of: a cardiac electrogram electrode, an RF generator power output sensor, an instrument distal portion impedance sensor, and an instrument distal portion force sensor; and wherein the controller is configured to affect the operation of the electromechanically steerable elongate catheter by automatically executing an instrument movement or treatment command based upon changes in information received from the treatment interactivity variable sensor.

### RELATED APPLICATION DATA

The present application claims the benefit under 35 U.S.C. §119 to U.S. Provisional Patent application Ser. No. 61/349,690, filed May 28, 2010. The foregoing application is hereby incorporated by reference into the present application in its entirety.

The present application is also related to application Ser. Nos. ______ (Attorney Docket No. HNMD-20072.00), ______ (Attorney Docket No. HNMD-20072.01), and ______ (Attorney Docket No. HNMD-20072.03), all of which are filed on the same date herewith. The disclosures of the foregoing applications are expressly incorporated herein by reference.

### FIELD OF THE INVENTION

The invention relates generally to the minimally invasive medical techniques, and more particularly to the automation of certain aspects of therapeutic treatments using instruments such as electromechanically or robotically operated catheters.

### BACKGROUND

Elongate medical instruments, such as catheters, are utilized in many types of medical interventions. Many such instruments are utilized in what have become known as “minimally invasive” diagnostic and interventional procedures, wherein small percutaneous incisions or natural orifices or utilized as entry points for instruments generally having minimized cross sectional profiles, to mitigate tissue trauma and enable access to and through small tissue structures. One of the challenges associated with minimizing the geometric constraints is retaining functionality and controllability. For example, some minimally invasive instruments designed to access the cavities of the blood vessels and/or heart have steerable distal portions or steerable distal tips, but may be relatively challenging to navigate through tortuous vascular pathways with varied tissue structure terrain due to their inherent compliance. Even smaller instruments, such as guidewires or distal protection devices for certain vascular and other interventions, may be difficult to position due to their relatively minimal navigation degrees of freedom from a proximal location, and the tortuous pathways through which operators attempt to navigate them. To provide additional navigation and operational functionality options for minimally invasive interventions, it is useful to have an instrument platform that may be remotely manipulated with precision, such as the robotic catheter system available from Hansen Medical, Inc. under the tradename Sensei®. It would be useful to have variations of such a platform that are configured for not only providing a navigable platform as an instrument or stepping off point for another associated instrument, but also configured to automate certain aspects of procedures of interest, such as RF ablation procedures, transseptal puncture or crossing procedures, and chronic total occlusion procedures.

### SUMMARY

One embodiment is directed to a robotic catheter system, comprising a controller including a master input device; and an electromechanically steerable elongate instrument having a proximal interface portion and a distal portion, the proximal interface portion being configured to be operatively coupled to an electromechanical instrument driver in communication with the controller, the distal portion being configured to be interactively navigated adjacent internal tissue structures of a patient's body in response to signals from the controller; wherein the controller is operatively coupled to a treatment interactivity variable sensor selected from the group consisting of: a cardiac electrogram electrode, an RF generator power output sensor, an instrument distal portion impedance sensor, and an instrument distal portion force sensor; and wherein the controller is configured to affect the operation of the electromechanically steerable elongate catheter by automatically executing an instrument movement or treatment command based upon changes in information received from said treatment interactivity variable sensor. The controller may be operatively coupled to a cardiac electrogram electrode, and upon sensing that the amplitude of a cardiac electrogram signal from the cardiac electrogram electrode has changed over time at a rate exceeding a predetermined threshold rate, the controller may be configured to move the instrument to another position or increase the rate of movement of the instrument. The controller may be operatively coupled to a cardiac electrogram electrode and an RF generator configured to transmit energy to the distal portion of the elongate instrument at a transmission rate to treat tissues adjacent thereto, and upon sensing that the amplitude of a cardiac electrogram signal from the cardiac electrogram electrode has changed over time at a rate exceeding a predetermined threshold rate, the controller may be configured to change the transmission rate. The controller may be operatively coupled to an RF generator configured to transmit energy to an electrode positioned at the distal portion of the elongate instrument at a transmission rate to treat tissues adjacent thereto, and upon sensing that the transmission rate has changed over time at a rate exceeding a predetermined threshold rate, the controller may be configured to move the instrument to another position or increase the rate of movement of the instrument. The controller may be operatively coupled to an RF generator configured to transmit energy to an electrode positioned at the distal portion of the elongate instrument at a transmission rate to treat tissues adjacent thereto, and upon sensing that the transmission rate has changed over time at a rate exceeding a predetermined threshold rate, the controller may be configured to change the transmission rate. The controller may be operatively coupled to an'impedance sensor coupled to the distal portion of the elongate instrument, and upon sensing that the impedance has changed over time at a rate exceeding a predetermined threshold rate, the controller may be configured to move the instrument to another position or increase the rate of movement of the instrument. The controller may be operatively coupled to an impedance sensor coupled to the distal portion of the elongate instrument and an RF generator configured to transmit energy to the distal portion of the elongate instrument at a transmission rate to treat tissues adjacent thereto, and upon sensing that the impedance has changed over time at a rate exceeding a predetermined threshold rate, the controller may be configured to change the transmission rate. The controller may be operatively coupled to force sensor configured to sense interfacial forces between the distal portion of the elongate instrument and adjacent structures, and upon sensing that a sensed force applied at a sensed vector has changed over time at a rate exceeding a predetermined threshold rate or exceeds a predetermined threshold value, the controller may be configured to move the instrument to another position in a direction opposite to the sensed force vector. The controller may be configured to continue moving the instrument in the direction opposite to the sensed forced vector until the sensed force vector decreases by a predetermined percentage, or to a preset threshold value.

Another embodiment is directed to a method for operating a robotic catheter system, comprising transmitting a movement command generated with a master input device to a controller, the controller being operatively coupled to an electromechanically steerable elongate instrument having a proximal interface portion and a distal portion, the proximal interface portion being configured to be operatively coupled to an electromechanical instrument driver in communication with the controller, the distal portion being configured to be interactively navigated adjacent internal tissue structures of a patient's body in response to signals from the controller; wherein the controller is operatively coupled to a treatment interactivity variable sensor selected from the group consisting of: a cardiac electrogram electrode, an RF generator power output sensor, an instrument distal portion impedance sensor, and an instrument distal portion force sensor; and wherein the controller is configured to affect the operation of the electromechanically steerable elongate catheter by automatically executing an instrument movement or treatment command based upon changes in information received from said treatment interactivity variable sensor. The controller may be operatively coupled to a cardiac electrogram electrode, wherein upon sensing that the amplitude of a cardiac electrogram signal from the cardiac electrogram electrode has changed over time at a rate exceeding a predetermined threshold rate, the controller is configured to move the instrument to another position or increase the rate of movement of the instrument. The controller may be operatively coupled to a cardiac electrogram electrode and an RF generator configured to transmit energy to the distal portion of the elongate instrument at a transmission rate to treat tissues adjacent thereto, wherein upon sensing that the amplitude of a cardiac electrogram signal from the cardiac electrogram electrode has changed over time at a rate exceeding a predetermined threshold rate, the controller is configured to change the transmission rate. The controller may be operatively coupled to an RF generator configured to transmit energy to an electrode positioned at the distal portion of the elongate instrument at a transmission rate to treat tissues adjacent thereto, wherein upon sensing that the transmission rate has changed over time at a rate exceeding a predetermined threshold rate, the controller is configured to move the instrument to another position or increase the rate of movement of the instrument. The controller may be operatively coupled to an RF generator configured to transmit energy to an electrode positioned at the distal portion of the elongate instrument at a transmission rate to treat tissues adjacent thereto, wherein upon sensing that the transmission rate has changed over time at a rate exceeding a predetermined threshold rate, the controller is configured to change the transmission rate. The controller may be operatively coupled to an impedance sensor coupled to the distal portion of the elongate instrument, wherein upon sensing that the impedance has changed over time at a rate exceeding a predetermined threshold rate, the controller is configured to move the instrument to another position or increase the rate of movement of the instrument. The controller may be operatively coupled to an impedance sensor coupled to the distal portion of the elongate instrument and an RF generator configured to transmit energy to the distal portion of the elongate instrument at a transmission rate to treat tissues adjacent thereto, wherein upon sensing that the impedance has changed over time at a rate exceeding a predetermined threshold rate, the controller is configured to change the transmission rate. The controller may be operatively coupled to force sensor configured to sense interfacial forces between the distal portion of the elongate instrument and adjacent structures, wherein upon sensing that a sensed force applied at a sensed vector has changed over time at a rate exceeding a predetermined threshold rate or exceeds a predetermined threshold value, the controller is configured to move the instrument to another position in a direction opposite to the sensed force vector. The controller may be configured to continue moving the instrument in the direction opposite to the sensed forced vector until the sensed force vector decreases by a predetermined percentage, or to a preset threshold value.

### BRIEF DESCRIPTION OF THE DRAWINGS

FIG. 1 illustrates a robotic catheter system configured for conducting minimally invasive medical interventions.

FIG. 2 illustrates an instrument driver and instrument assembly of a robotic catheter system configured for conducting minimally invasive medical interventions.

FIG. 3A illustrates a distal portion of an instrument assembly configured for conducting ablation treatments.

FIG. 3B illustrates a distal portion of an instrument assembly configured for conducting treatments involving the traversal of a needle or wire-like instrument through at least a portion of a tissue structure.

FIG. 3C illustrates a distal portion of an instrument assembly configured for conducting treatments involving the traversal of a scalpel type instrument portion through at least a portion of a tissue structure.

FIGS. 4A-4D illustrate various embodiments for detecting an amount of instrument traversal into a tissue structure.

FIG. 4E illustrates an instrument assembly wherein a scalpel tip is coupled to the remainder of the assembly with a joint.

FIG. 5 illustrates a cardiac ablation scenario employing an instrument assembly configured to sense temperature and load.

FIGS. 6A and 6B illustrate views of a user interface configured to facilitate customization of a tissue contact scenario.

FIG. 7 depicts a flow chart illustrating various aspects of an ablation treatment.

FIGS. 8A-8C illustrate a tissue structure puncturing scenario employing an instrument assembly configured to sense loads of various aspects of the assembly.

FIG. 9 depicts a flow chart illustrating various aspects of a tissue wall traversal treatment.

FIGS. 10A-10C illustrate a structure traversing scenario employing an instrument assembly configured to sense loads of various aspects of the assembly.

FIG. 10D depicts a cross sectional view of structures depicted in FIG. 10C.

FIG. 10E illustrates an interventional planning scenario.

FIG. 11 depicts a flow chart illustrating various aspects of a structure traversal treatment.

FIG. 12 depicts a flow chart illustrating various aspects of a treatment interactivity variable based treatment.

FIGS. 13A and 13B illustrate plots of scaling versus detected force which may be utilized in accordance with the present invention.

FIG. 14 illustrates a haptic overlay plotting in accordance with one embodiment.

FIGS. 15A and 15B illustrate one embodiment of an instrument assembly in accordance with the present invention which comprises a plurality of strain gauges to detect a force vector.

FIGS. 16A-16G illustrate one embodiment of a tissue intervention procedure in accordance with the present invention.

FIGS. 17A and 17B illustrate aspects of one embodiment of an intervention paradigm wherein a zig zag type pattern is utilized to create a substantially curvilinear lesion.

FIG. 18 depicts a flow chart illustrating various aspects of a multifactorial treatment technique in accordance with the present invention.

FIGS. 19A-19C depict graphical representations of three relationships which may be utilized to vary master input device motion scaling.

### DETAILED DESCRIPTION

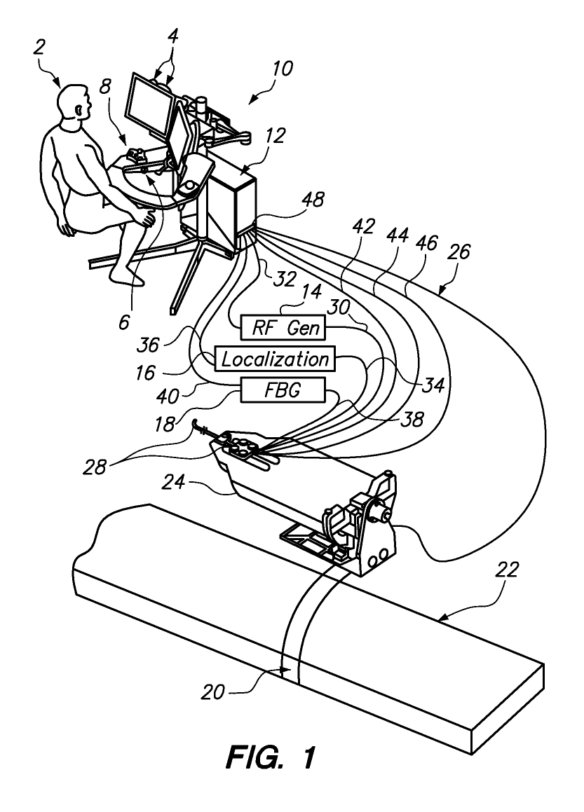

Referring to FIG. 1, a robotic catheter system is depicted having an operator workstation (10) comprising a master input device (6), control button console (8), and a display (4) for the operator (2) to engage. In the depicted embodiment, a controller or control computer configured to operate the various aspects of the system is also located near the operator (2). The controller (12) comprises an electronic interface, or bus (48), configured to operatively couple the controller (12) with other components, such as an electromechanical instrument driver (24), RF generator (14), localization system (16), or fiber bragg shape sensing and/or localization system (18), generally via electronic leads (32, 30, 36, 34, 40, 38, 42, 44, 46, 26). Electromechanically or robotically controlled catheter systems similar to that depicted in FIG. 1 are available from Hansen Medical, Inc. under the tradename Sensei®, and described, for example, in U.S. patent application Ser. Nos. 11/481,433, 11/073,363, 11/678,001 (“Intellisense”), and 11/637,951, each of which is incorporated by reference in its entirety. In the depicted embodiment, the controller (12) preferably is operatively coupled (32) to the RF generator (14) and configured to control outputs of the RF generator (14), which may be dispatched via electronic lead (30) to the disposable instrument assembly (28). Similarly, the controller (12) preferably is operatively coupled (36) to a localization system, such as an electromagnetic or potential difference based localization system (16), such as those available under the tradenames CartoXP® and EnSite® from Biosense Webster, Inc., and St. Jude Medical, Inc., respectively. The localization system (16) preferably is operatively coupled via one or more leads (34) to the instrument assembly (28), and is configured to determine the three dimensional spatial position, and in certain embodiments orientation, of one or more sensors coupled to a distal portion of the instrument assembly relative to a coordinate system relevant to the controller and operator, such as a world coordinate system. Such position and/or orientation information may be communicated back to the controller (12) via the depicted electronic lead (36) or other signal communication configuration such as a wireless data communication system (not shown), to enable the controller (12) and operator (2) to understand where the distal portion of the instrument assembly (28) is in space—for control and safety purposes. Similarly, a fiber bragg localization and/or shape sensing system (18) may be coupled between the controller (12) and instrument assembly (28) to assist with the determination of position and shape of portions of the instrument assembly, thermal sensing, contact sensing, and load sensing, as described, for example, in the aforementioned incorporated patent applications. In one embodiment, a fiber (38) comprising Bragg gratings may be positioned between the distal tip of one or more instruments in the assembly and coupled proximally to the fiber bragg analysis system (18), and outputs from such system may be electronically communicated (40) to the controller (12) to facilitate control and safety features, such as closed loop shape control of one or more portions of the instrument assembly, as described, for example, in the aforementioned incorporated references. A feedback and control lead (26) is utilized to operatively couple the instrument driver (24) to the controller. This lead (26) carries control signals from the controller (12) to various components comprising the instrument driver (24), such as electric motors, and carries control signals from the various components of the instrument driver (24), such as encoder and other sensor signals, to the controller (12). The instrument driver (24) is coupled to the operating table (22) by a setup structure (20) which may be a simple structural member, as depicted, or a more complicated movable assembly, as described in the aforementioned incorporated references.

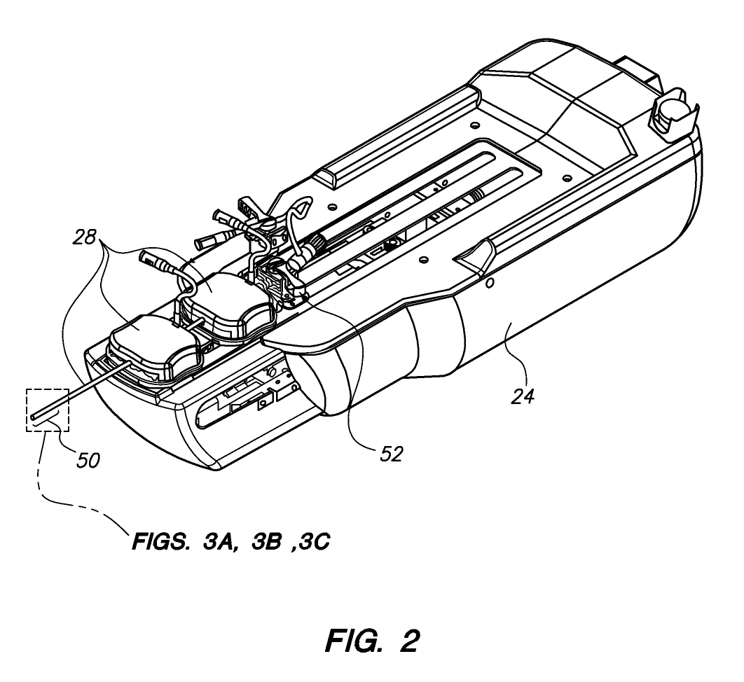

Referring to FIG. 2, a close orthogonal view of an instrument driver (24) and instrument assembly (28) is depicted, this configuration having the ability to monitor loads applied to working members or tools placed through a working lumen defined by the instrument assembly (28). In this embodiment, such loads are determined with load sensors (52) located within the housing of the instrument driver, as described in the aforementioned incorporated references. In other embodiments, loads imparted to various tools or aspects of the instrument assembly (28) may be monitored using load sensors or components thereof which are embedded within or coupled to distal portions (50) of such tools or instrument assembly portions.

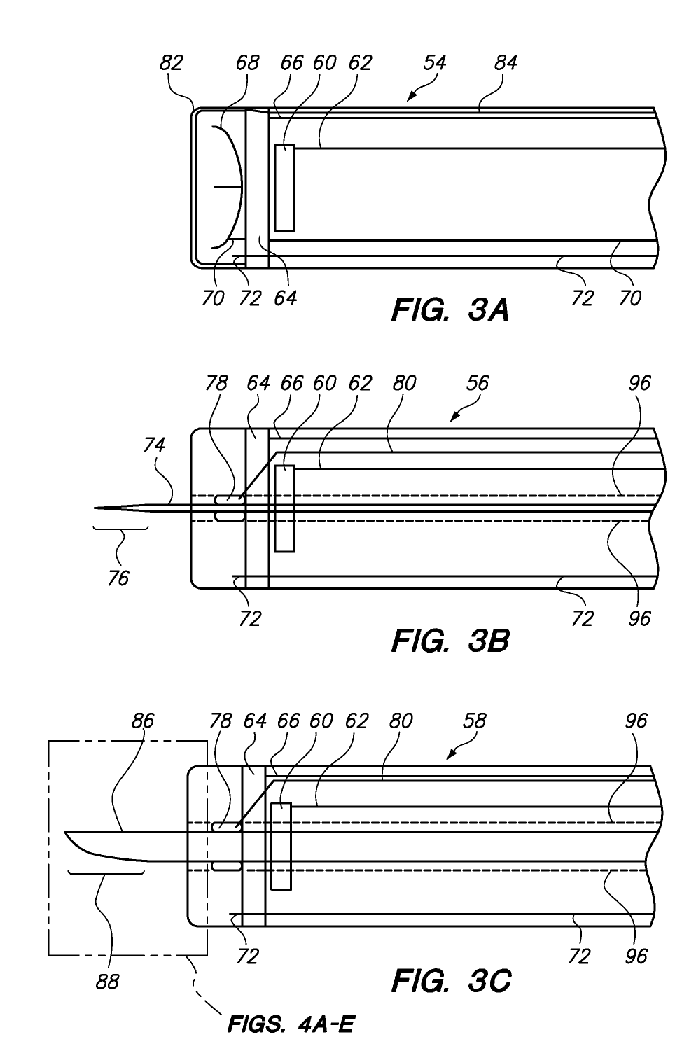

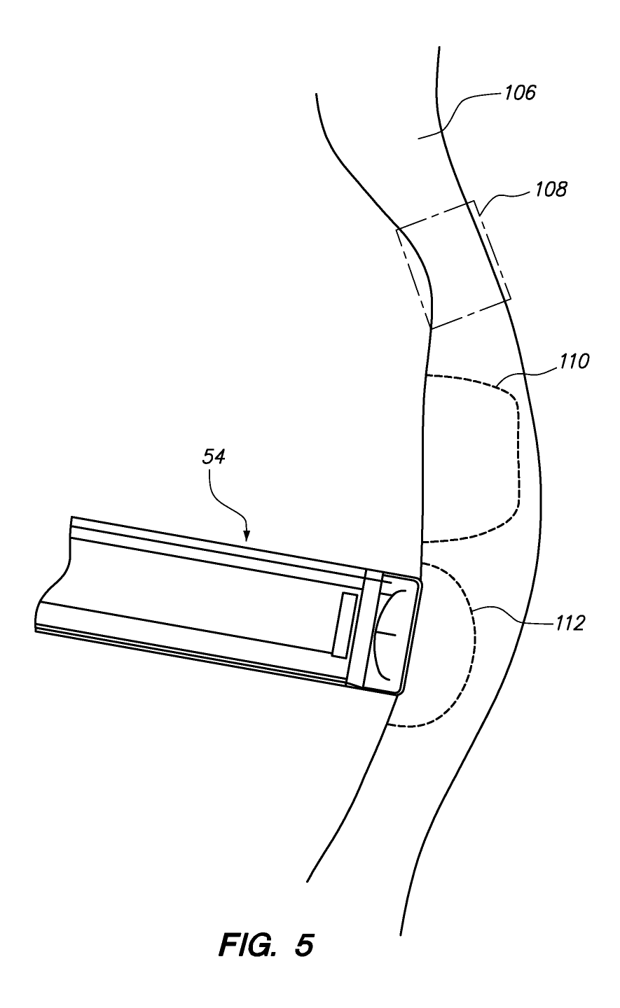

Referring to FIG. 3A, an instrument assembly distal portion (54) configured for ablation therapy is depicted, comprising a distally located RF electrode (82) coupled to an RF generator (not shown in FIG. 3A; element 14 of FIG. 1). The depicted embodiment comprises a microwave, antenna (68) distally coupled to the instrument portion and electronically coupled via a lead (70) back to the controller (not shown in FIG. 3A; element 12 of FIG. 1). Further, the depicted embodiment comprises a load sensor (64) mechanically positioned to sense loads applied to the most distal portion of the instrument assembly (54). Signals associated with loads are communicated via a lead (66) back to the controller for interpretation and analysis. The load sensor may, for example, comprise one or more strain gauges of various types, one or more localization sensors with a deflectable member of known spring constant in between, one or more fiber bragg sensors with fibers or other associated deflectable members of known spring constant, and/or movable fluid reservoir type pressure/load sensors. Further, the embodiment of FIG. 3A may comprise one or more localization sensors (60) coupled via an electronic lead (62) to a localization system, as well as a fiber bragg shape and/or deflection sensing fiber (72) configured to assist in the determination of shape and bending deflection of the instrument assembly portion (54). In one embodiment, the microwave antenna (68) may be utilized to conduct radiometry analysis, such as black body radiometry analysis, of nearby structures, such as heated tissue structures, as described, for example in U.S. Pat. Nos. 5,683,382 and 6,932,776, both of which are incorporated by reference herein in their entirety. Utilization of such an embodiment is described below in reference to FIGS. 5, 6, and 7.

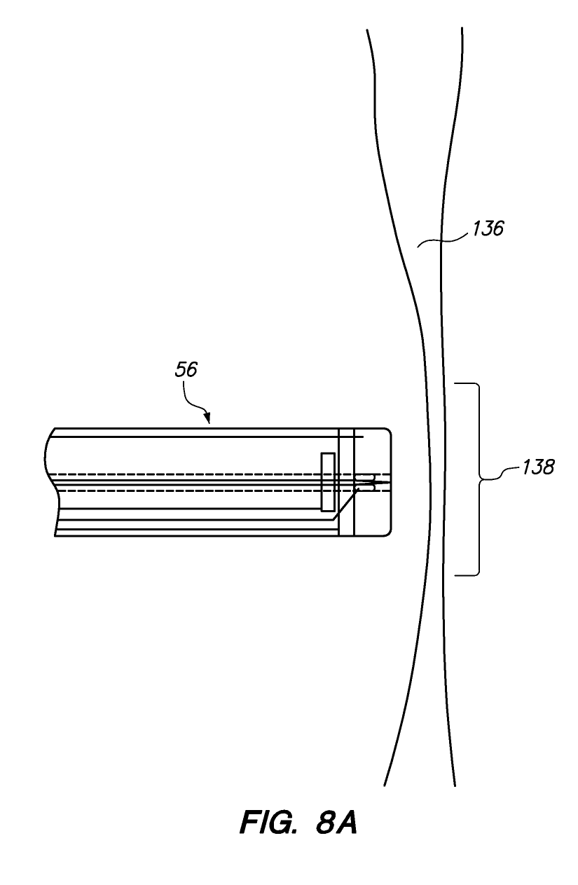

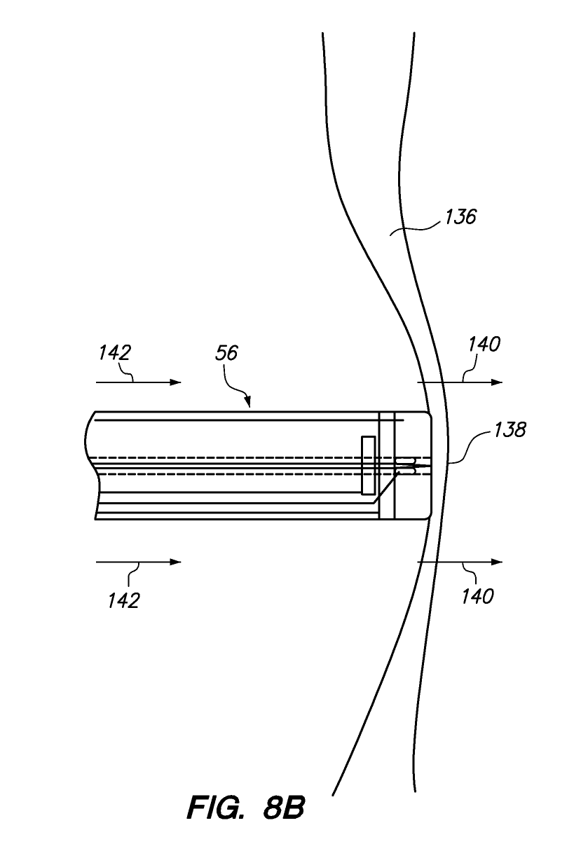

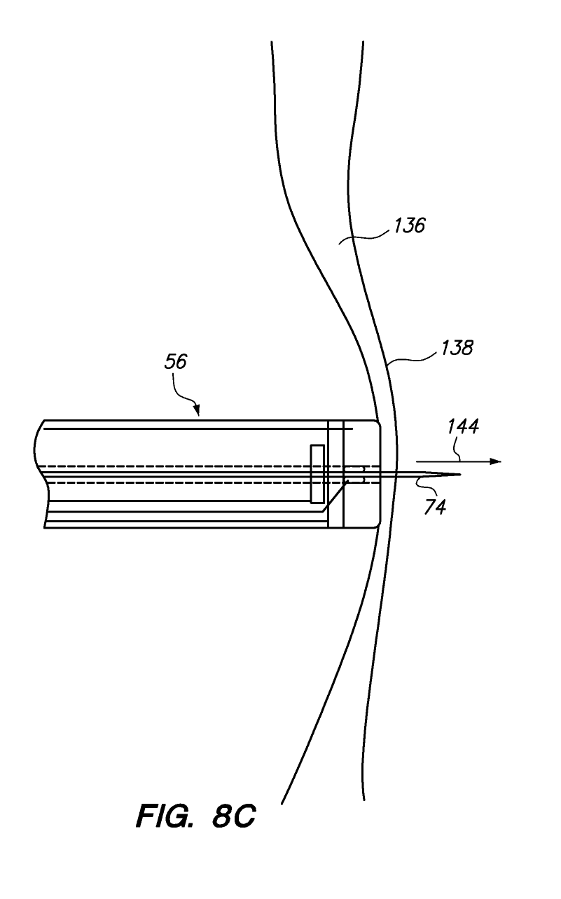

Referring to FIG. 3B, another embodiment of an instrument assembly distal portion (56) is depicted, this embodiment being configured for traversing or piercing a nearby structure, such as a tissue wall or endovascular plaque structure. As shown in FIG. 3B, the instrument assembly may comprise a load sensor (64), localization sensor (60), and fiber bragg sensor (72), as with the embodiment of FIG. 3A. A working lumen (96) is defined through the center of the assembly to accommodate a slender traversing tool (74), such as a wire, guidewire, or needle, which in the depicted embodiment has a sharpened tip (76). The traversing tool (74) and working lumen (96) are sized to allow relative motion, such as rotational and/or translational motion, between the lumen and tool, and in the depicted embodiment, a braking mechanism is included to prevent relative motion between the two, such as in certain traversing scenarios, or situations wherein it is desirable to transfer loads imparted upon the traversing tool (74) to the very distal portion of the instrument assembly so that the load sensor (64) will read such loads. In the depicted embodiment, the braking mechanism comprises a controllably inflatable annular balloon (78) which may be remotely inflated using a fluid lumen (80). Utilization of such an embodiment is described below in reference to FIGS. 8A through 11.

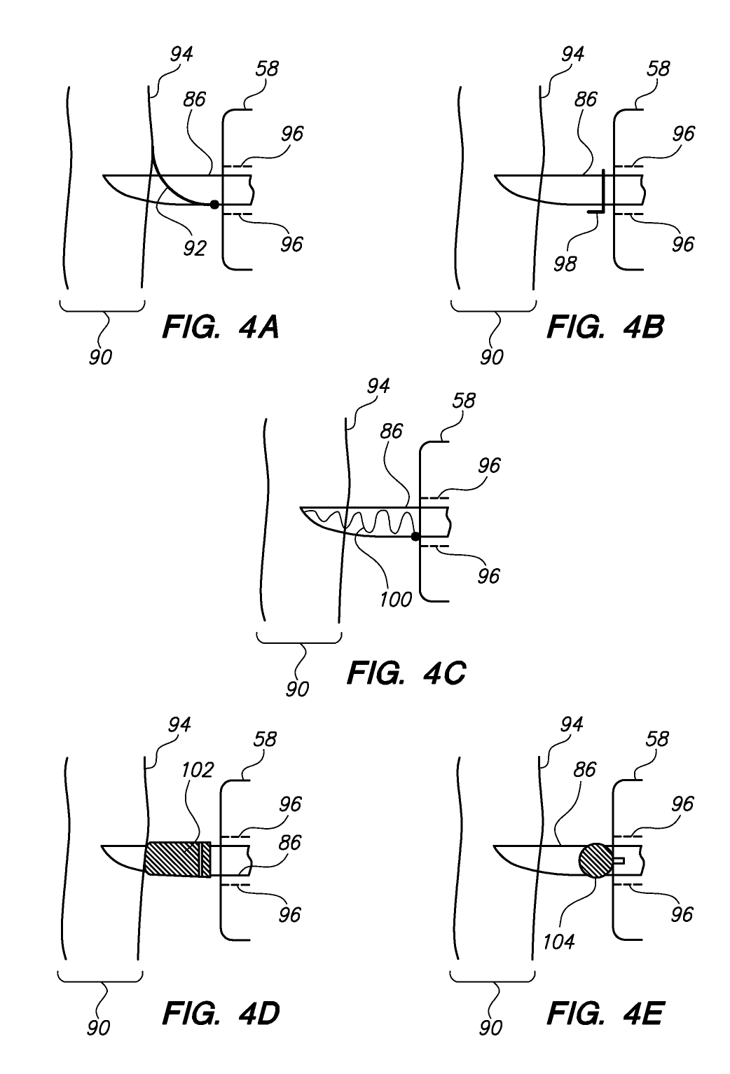

Referring to FIG. 3C, an instrument assembly distal portion (58) embodiment similar to that depicted in FIG. 3B is depicted, with the exception that in the embodiment of FIG. 3C, the working lumen (96) is larger and the traversing tool (86) comprises a scalpel cutting tip (88). Referring to FIG. 4E, in one embodiment, it is desirable to have a jointed coupling (104) between the proximal and distal portions of the scalpel tipped traversing tool (86) to facilitate automatic following of the traversing or cutting surface with motion of the instrument assembly (58) as the nearby tissue structure (90) and surface thereof (94) is being cut or traversed.

Referring to FIGS. 4A-4D, four variations of traversal depth sensing configurations are depicted which may be used with scalpel, needle, wire, or other type traversing tools to determine how much of such tool has been extended or traversed into the subject tissue structure (90), past the tissue structure outer surface (94). Referring to FIG. 4A, in one embodiment, a flexible follower member (92) may be configured to bend through contact with the tissue structure (90) surface (94) as the traversing tool (86) is inserted past the surface (94). A bending sensor, such as a fiber bragg sensing fiber, strain gauge, or the like may be utilized along with known mechanics of such follower member (92) to determine how much the traversing tool (86) has extended into the tissue structure (90) past the surface (94). In another embodiment (not shown), the follower member may be rigid, and may rotate along with an encoder or other rotation sensor relative to the traversing tool (86), to allow for determination of traversal depth without flexion of the follower member. Referring to FIG. 4B, a proximity sensor may be coupled to the traversing tool (86) and configured to transmit and receive reflected sound, light, or other radiation from the surface (94) to determine the traversal depth. Referring to FIG. 4C, a surface contact sensor (100), such as one based upon an electronic lead coupled to the surface of the traversing tool (86) tip, may be utilized to sense traversal depth through direct contact with the traversed portions of the tissue structure (90). Referring to FIG. 4D, a collar (102) may be configured to slide relative to the traversing tool (86) and remain at the surface (94) of the tissue structure (90), while a sensor such as a linear potentiometer may be utilized to determine how much the end of the collar (102) has moved relative to the end of the traversing tool (86), for determination of traversal depth.

Referring to FIG. 5, an embodiment such as that depicted in FIGS. 1, 2, and 3A is illustrated in situ adjacent a tissue structure (106) such as a heart cavity wall. In one embodiment, one or more medical imaging modalities, such as computed tomography (“CT”), magnetic resonance (“MRI”), or ultrasound, preferably are utilized preoperatively to understand the pertinent anatomy. Images from such modalities may be filtered and/or segmented to produce two or three dimensional surface models with which preoperative or intraoperative planning and instrument navigation may be conducted. In one embodiment, an operator may preoperatively mark certain portions of the tissue structure (106) as zones where contact should be avoided—these may be called “keep out zones” and labeled in a graphical user interface presented to the operator on a display as a dashed box (108), or otherwise highlighted area, and preferably the associated robotic catheter system controller is configured to not allow an instrument assembly which has a control system registered to such images and keep away zones (108) to move the distal portion of such instrument assembly (54) into such zone (106). In one embodiment, for example, such zones may be placed at thin walled areas, areas known to be at risk for possible fistulas, or areas of previous tissue damage or therapy. Indeed, in the depicted embodiment, a slightly different marker (110) is utilized to depict in the graphical user interface a previously heated or ablated volume. In one embodiment, volumes which have received previous therapy may be marked with graduations in color, shading, and/or highlighting to indicate different graduations of therapy. For example, cardiac muscle conduction blockage is generally associated with collagen denaturation of the such tissue. Such collagen denaturation can be created with applied heat, such as that applied with RF energy in an RF ablation procedure. In one embodiment, the operator may configure the controller to avoid volumes with the instruments which are known to have been heated at all. In another embodiment, the controller may be configured to only allow contact and associated delivery of RF energy to volumes known to have not received adequate energy for denaturation, and to stop the delivery of energy past a certain level of temperature and/or associated denaturation. Preferably the microwave antenna (shown as element 68 in FIG. 3A) is utilized to determine the temperature of associated tissues in real or near-real time, along with microwave radiometry computer software operated by the controller (12) computer or other computing system, and preferably such temperature is depicted graphically (112) for the operator using gradients of colors, shading, and/or highlighting in real or near real time, to facilitate an actively monitored precision thermal intervention while the RF generator may be utilized to cause the RF electrode tip to emit RF energy to the adjacent tissue structure portion. In other words, RF may be used to interactively heat the tissue, and microwave radiometry may be utilized to observe the heating and/or modify the variables of the intervention, such as RF power, timing of RF emission, movement of the RF electrode, and the like. In one embodiment, a thermodynamic model may be utilized to understand the heating dynamics of the instrument and associated tissues. For example, preoperatively and/or intraoperatively, Doppler ultrasound analysis may be utilized along with the aforementioned anatomical images to map flow through the cardiac cavities, flow through the nearby vessels and sinuses, tissue density, tissue structure local thickness/volume and ability to handle and dissipate heat, and other factors pertinent to the denaturation conduction block electrophysiology therapy model. Computational fluid dynamics may be utilized to create thermodynamic models pertinent to localized RF-heat-based denaturation. In another embodiment, tissue structure thickness, volume, and thermal inertia qualities may be examined by applying small amounts of RF energy, such as enough to heat a nearby tissue structure portion by about ten percent, and watching the decay of temperatures after such heating.

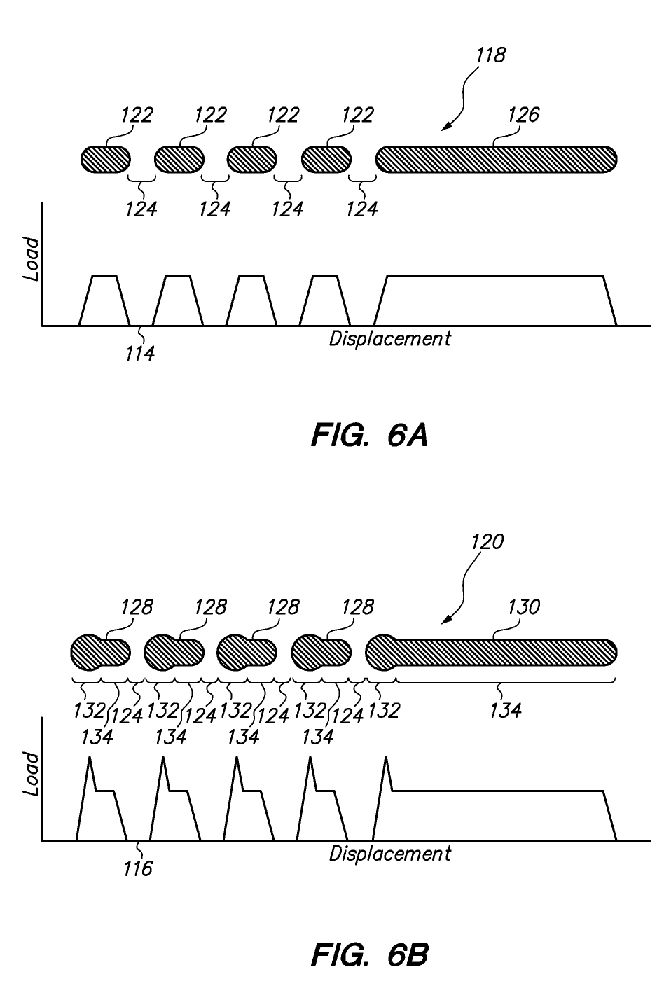

It has been found in various scientific studies that contact load is an important variable in RF-heat-based denaturation of cardiac tissue for aberrant conduction pathway blockage. Preferably the inventive system may be configured to customize many aspects of the physical contact scenario between instruments and tissue structures. For example, referring to FIG. 6A, a graphical user interface control panel preferably is configured to allow an operator to custom tailor a contact scenario between instrument and tissue structure. A load-displacement graphical representation (118) is depicted alongside a plot of load versus displacement (114), and the operator is able to make adjustments through the graphical user interface to both. In the variation depicted in FIG. 6A, the operator has configured the instrument to have four intermittent bouts of contact and dragging with the tissue structure, followed by a longer-in-distance bout of contact/dragging. The associated plot of load versus displacement (114) shows that as the instrument is placed into contact for each of the short (122) and long (126) drags, the load is taken up to a prescribed load amount and held until the end of such drag, after which the load goes to zero during one of the gaps in contact (124) between the instrument and tissue structure. This scenario is somewhat akin to drawing a dashed and then solid line with a pencil on a piece of paper—but in the subject clinical/instrumentation scenario, an RF electrode would be creating such a pattern on a selected tissue structure surface. Referring to FIG. 6B, a contact configuration similar to that depicted in FIG. 6A is depicted, with the exception that the operator has configured the instrument to start each drag (128, 120) with an impulse of relatively higher load, and then to taper back to the load seen in the variation of FIG. 6A for the remainder of each drag. The loading variations may be depicted in the load-displacement graphical representation (120) with the relatively high load drag portions (132) being highlighted with larger marking, and the remaining relatively low drag portions (134) being highlighted as in FIG. 6A. The load versus displacement plot (116) is further illustrative of the loading and contact scenario. Again, there is a useful analogy to using a pencil on a piece of paper. One can see that many variations in loading, intermittence, and dragging patterns may be created and executed with such a control interface, to control not only contact, but also loads of contact, during interventional procedures.

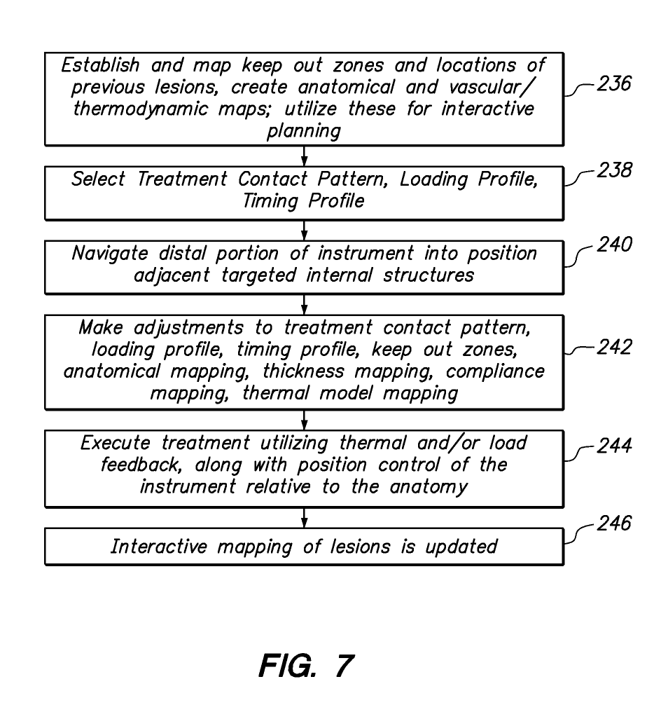

Referring to FIG. 7, various aspects of embodiments of treatment paradigms utilizing configurations such as those depicted in FIGS. 1, 2, 3A, 5, 6A, and 6B are illustrated with a flow chart. As shown in FIG. 7, preoperative (or in another embodiment intraoperative) imaging studies may be utilized to map the anatomy, vasculature, and flow patterns. This information may be utilized to create thermodynamic models of portions of the tissue structure of interest. Further, keep out zones may be flagged using previous intervention data or imaging data. All of this information may be utilized for interactive planning purposes (236) along with three dimensional instrument simulation techniques described for the subject robotic catheter system in the aforementioned incorporated references. Next (238) an operator may select a treatment contact pattern for various planned lesions, as described, for example, in FIGS. 6A and 6B. A timing profile, including time to be spent at each location and related dragging velocity, may also be prescribed. Such a timing profile may be influenced by the models created in the previous step (236), such as tissue structure wall thickness and thermodynamic models. Intraoperatively, the instrument assembly may be navigated, such as by a robotic instrument driver, to desired positions adjacent targeted internal structures (240). Such navigation may be accomplished using open loop kinematic-based position control, or closed loop position control using sensor information from devices such as a fiber bragg shape and/or localization sensing configuration or localization system, as described above in reference to FIG. 1, and in the aforementioned incorporated references. Given access to the anatomy intraoperatively, adjustments may be made to the treatment contact pattern, loading profile, timing profile, keep out zones, anatomical mapping, thickness mapping, compliance mapping, thermal model mapping, and general locations of desired contact between the instrument and anatomy (242). Subsequently the operator may execute the treatment (244) either manually or automatically using the robotic catheter system and a prescribed trajectory/position plan. Navigation may be controlled with position and/or load feedback using load sensors such as those described in relation to FIG. 3A or 2. A reference frame of a load sensor preferably is registered to a reference frame utilized by an operator to navigate the elongate instrument, such as a reference frame of a master input device or display utilized by the operator to visualize movement of the elongate instrument. New lesions preferably are observed in real or near-real time, as described in reference to FIG. 5, and are mapped onto an updated lesion mapping.

Referring to FIGS. 8A-8C, various aspects of a traversal intervention are illustrated, whereby an instrument or portion thereof may be controllably passed, or traversed, through at least a portion of a tissue structure. Referring to FIG. 8A, a tissue structure (136) wall is depicted having a thinned region (138), which may, for example, represent a fossa ovalis portion of an atrial cardiac septum, which may be desirably traversed for a trans-septal procedure wherein instruments are to be utilized in the left atrium of the heart. The instrument assembly portion (56) depicted has been advanced toward the tissue structure (136) but has not yet contacted such tissue structure. Referring to FIG. 8B, the instrument assembly (56) has been advanced (142) into contact with the targeted region (138) of the tissue structure (136), and this instrument advancement has caused a repositioning (140) and tensioning of the tissue structure, which may be, called “tenting” of the tissue structure. Tenting may be desirable to assist with positioning and vectoring the instrument assembly distal portion (56) and to temporarily alter the mechanical properties of the tissue structure (for example, in tension, a thinned wall is not as likely to continue to deform and move away from the instrument assembly when a traversing instrument is advanced toward and into such wall relative to the rest of the instrument assembly; the viscoelastic performance may also be desirably altered by placing the structure under tented loading). Referring to FIG. 8C, with the instrument assembly distal portion continuing to tent the targeted portion (138) of the tissue structure, the traversing member (74) may be inserted through the tissue structure. In one embodiment, such insertion may be conducted manually with a needle, guidewire, or similar working tool that extends proximally to a position wherein it may be manually manipulated by the hand of an operator. In another embodiment, insertion and retraction of such tool are controlled and actuated electromechanically, utilizing proximally positioned actuation mechanisms such as those disclosed in the aforementioned incorporated references, or by proximally triggered but distally actuated (such as by a spring or other stored energy source) mechanisms, such as those described in U.S. Pat. Nos. 4,601,710, 4,654,030, and 5,474,539, each of which is incorporated by reference herein in its entirety.

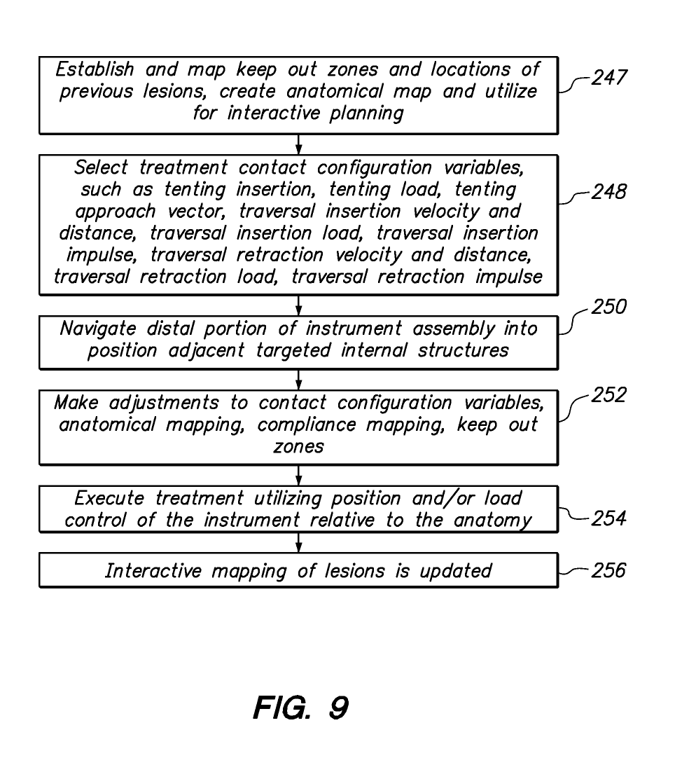

Referring to FIG. 9, a flowchart illustrates aspects of procedural embodiments for conducting a tissue traversal intervention. As shown in FIG. 9, in a similar manner as described in reference to FIG. 7, the system may be utilized along with preoperative imaging data to establish and map keep out zones and locations of previous lesions, for interactive planning purposes (247). The operator may configure the system with contact configuration variables such as tenting insertion load, velocity and impulse of tenting insertion, tenting approach vector with the instrumentation, traversal instrument velocity profile, traversal distance, traversal impulse and load profile, as well as traversal retraction velocity and distance, and traversal retraction load and impulse profile variables (248). The instrument assembly may be navigated into position adjacent targeted internal tissue structures (250), and adjustments may be made intraoperatively to contact configuration variables, anatomical mapping (such as with greater understanding of the thickness of various structures utilizing intraoperative imaging modalities such as in-situ instrument-based ultrasound), tissue structure compliance mapping, and keep out zones. Thickness mapping may be conducted using preoperative imaging to determine internal and external surface positions of various structures, or direct measurement of thicknesses from preoperative images. This information may be combined with further information gained from in-situ imaging techniques to increase the understanding of thickness, and also compliance of the tissue, as imaging and physical interaction may be utilized to understand compliance and density related variables, as described, for example, in the aforementioned incorporated references. Treatment may then be executed utilizing position and/or load control of the instrument portions relative to the anatomy, in accordance with the predetermined contact configuration variables (254), and the interactive mapping of lesions updated (256).

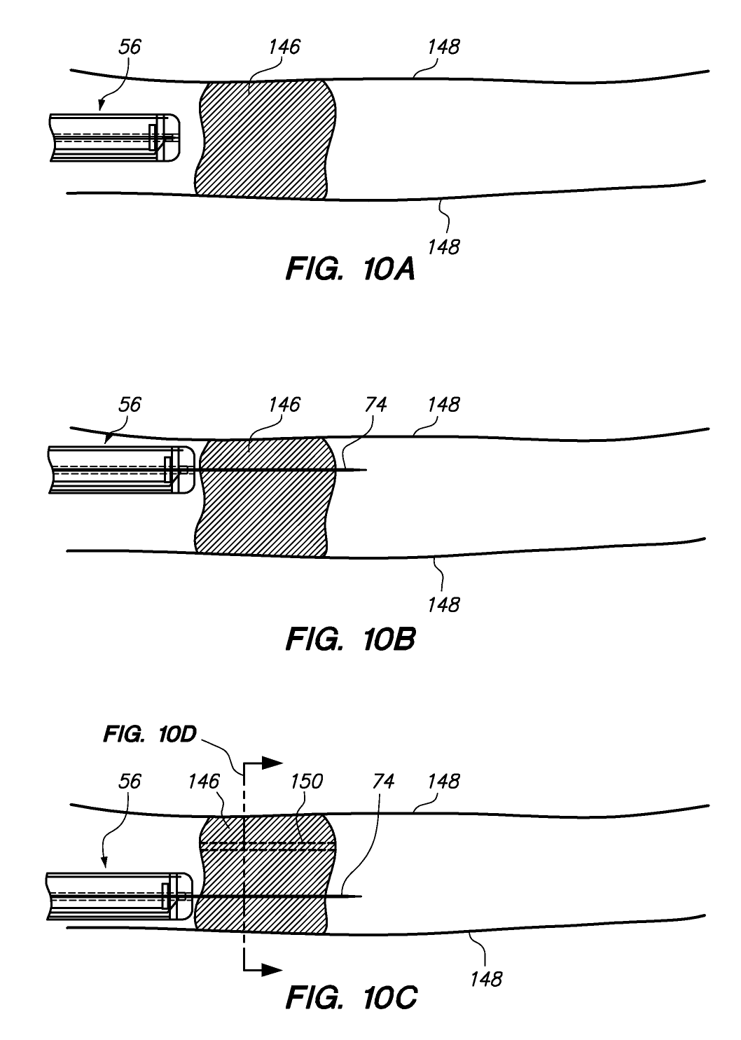

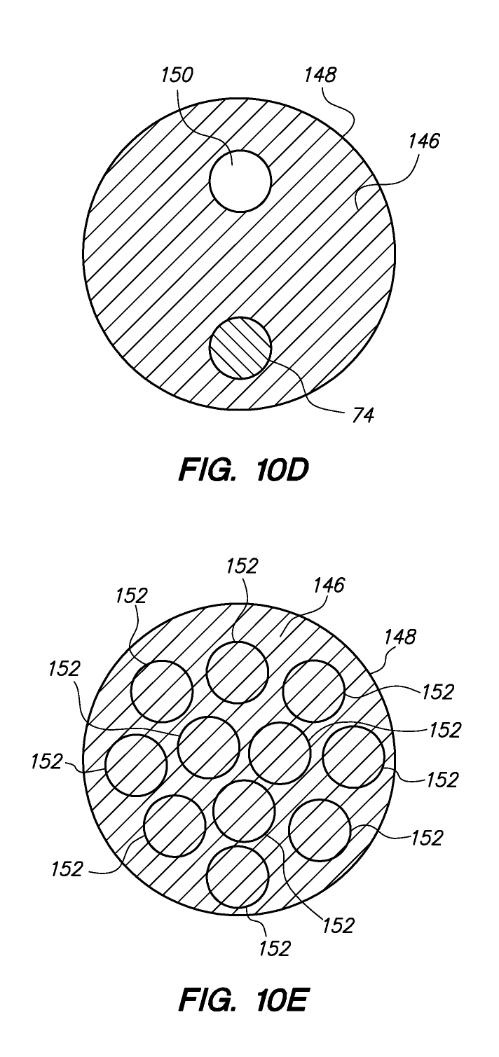

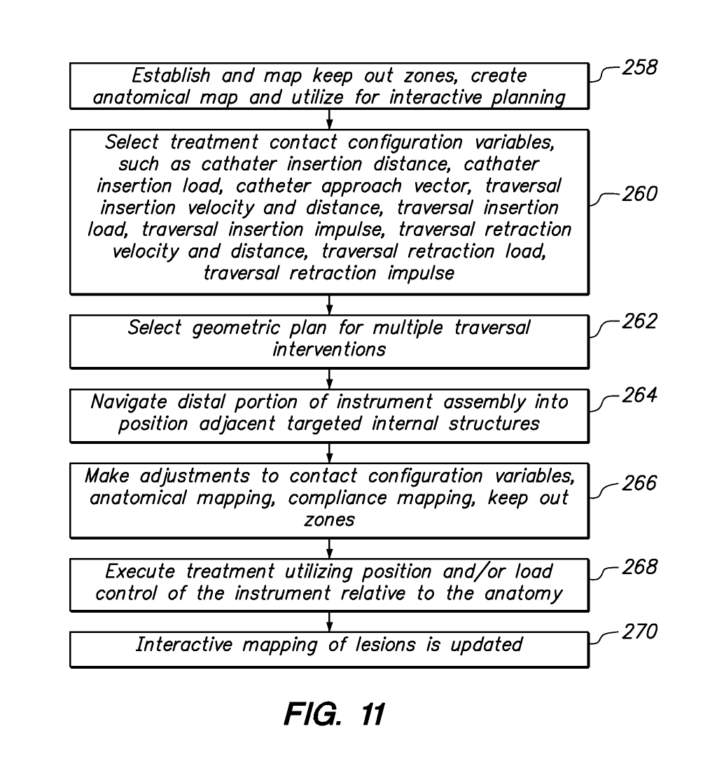

Referring to FIGS. 10A-10C and 11, various aspects of another traversal intervention are illustrated, featuring a traversal of an endovascular plaque, such as in a clinical condition known as chronic total occlusion, or “CTO”. Referring to FIG. 10A, a vascular plaque (146) structure occluding a vessel (148) is approached by an endovascular instrument assembly (56) configured for traversal. In a manner similar to that described in reference to FIG. 9, the instrument assembly may be configured to approach, establish contact with, and traverse, with a traversing tool (74) the plaque, as shown in FIG. 10B. Subsequently, the tool (74) may be retracted leaving a defect (150) in the plaque structure (146), the instrument assembly moved to a different location, and the plaque structure (146) readdressed and re-traversed with the traversing tool (74). FIG. 10D depicts a cross sectional view of the activities illustrated in FIG. 10C. Continued traversal may lead to dissolution or removal of the plaque, and referring to FIG. 10E, a pattern of planned traversal defects (152) preferably may be preoperatively or intraoperatively planned utilizing images of the anatomy and an understanding of the geometry of the traversing tool.

Referring to FIG. 11, a flowchart illustrates aspects of procedural embodiments for conducting a tissue traversal intervention; there are analogies to the procedures described in reference to FIGS. 7 and 9. As shown in FIG. 11, keep out zones may be established, an preoperative images may be utilized for interactive planning (258). Treatment contact configuration variables may be selected, such as the larger instrument subassembly (such as a catheter) insertion loads, velocity, approach vector, and the like (260). A geometric plan may be created for multiple traversals (262), as described above in reference to FIG. 10E. The instrument assembly may then be navigated into position adjacent the targeted internal structures, such as vascular plaque structures (264), adjustments made intraoperatively (266), and the treatment executed using position and/or load control of the instrument portions relative to the anatomy (268). Then the interactive mapping of lesions, or destruction of lesions or structures, may be updated (270).

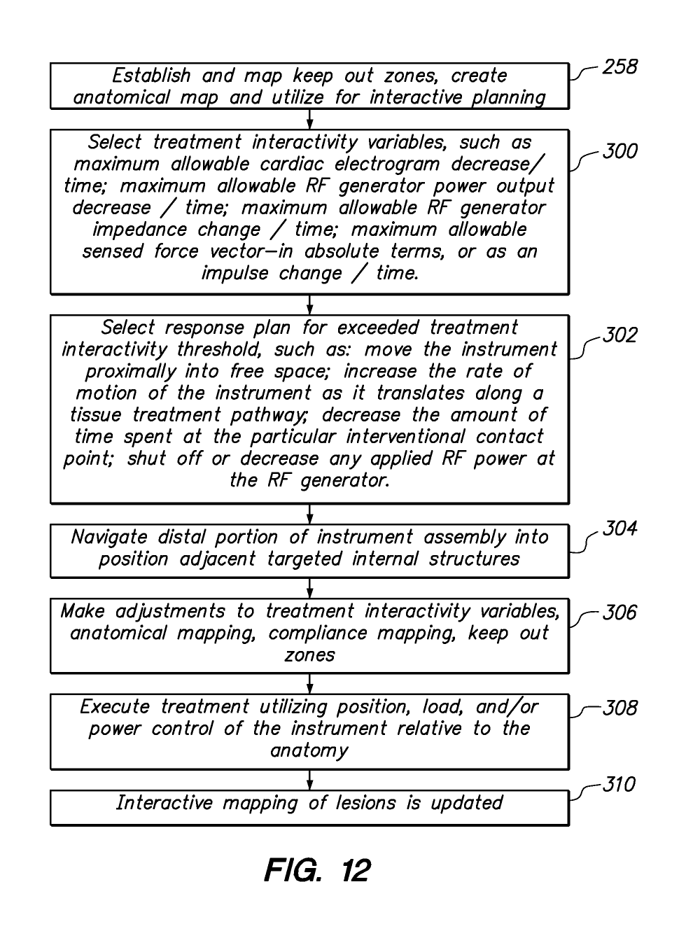

Referring to FIG. 12, in another embodiment, treatment interactivity variables may be utilized in automated operation of an electromechanical interventional instrument system. Referring to FIG. 12, subsequent to establishing and mapping keep out zones, creating an anatomical map for planning and the like (258), treatment interactivity variables may be selected (300) to match a particular hardware configuration, such as maximum allowable cardiac electrogram amplitude changes versus time in a hardware configuration featuring a cardiac electrogram sensor (such as one located distally on an elongate instrument), maximum allowable RF generator power output changes versus time in a hardware configuration featuring an RF generator which may be coupled to a distal treatment electrode, maximum allowable RF generator impedance change versus time in a hardware configuration featuring and RF generator and impedance monitoring capabilities, and maximum allowable sensed force vectors in absolute terms or as force change versus time (i.e., impulse) in hardware configurations wherein one or more force sensors may be utilized to detect loads imparted to an elongate instrument by surrounding structures, such as tissues or other instruments. A response plan paradigm may then be selected to direct a controller configured to operate the electromechanical elongate instrument in the instances wherein thresholds, such as those described above, are exceeded (302). For example, when a given threshold is exceeded, the controller may be configured to direct the instrument to move proximally into free space, to increase the rate of motion of the instrument as it translates adjacent or against the subject anatomy, to decrease the amount of time spent at any particular interventional contact location, or to shut off or decrease any applied RF power or other energy based treatment at its generator. Subsequently, the instrument distal portion navigation may be continued (304), adjustments may be made to operational variables (306), treatments may be executed (308), and interactive mapping of lesions continued (310).

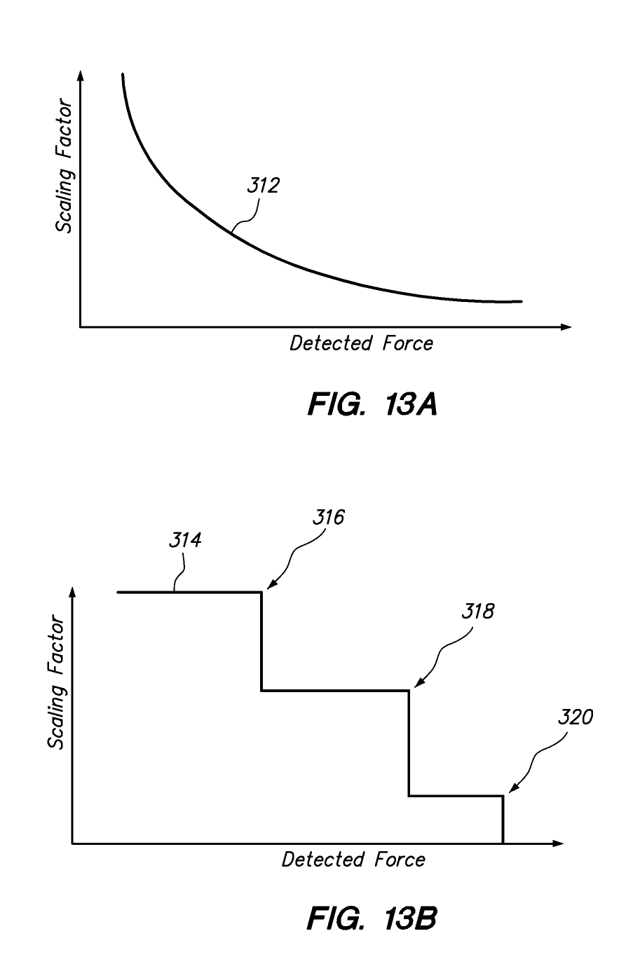

As described above, various embodiments of the subject elongate instrument assemblies may comprise load or force sensing devices, such as those featuring strain gauges, fiber bragg sensors, or the like, as described above, or proximal interfacial load sensing assemblies such as that sold by Hansen Medical, Inc. under the tradename “Intellisense”®. Any of these configurations may be utilized by a robotic instrument controller to modify a scaling ratio associated with a master input device configured to allow an operator to move an instrument. For example, in one embodiment, at relatively minimal or nonexistent detected forces, such as positions of the elongate instrument wherein the distal tip is in free space, the control system may be configured to move the instrument distal tip at a scaling ratio, such as 1:1, relative to master input device moves that the instrument is following. With larger detected forces, such scaling ratio may be decreased with a linear, curvilinear, or stepwise relationship, down to levels such as 1:0.5, 1:0.25, or less, to ensure that the instrument is moving in small increments relative to larger incremental commanded moves as the master input device when in the presence of other objects, such as tissue structures, as sensed through the force sensor. For example, a curvilinear relationship is illustrated by the plot (312) of FIG. 13A. In accordance with such an embodiment, for example, a master-slave instrument being operated in free space would move with a significantly greater scaling factor of master move relative to slave move, as compared with the same master/slave configuration moving in a scenario wherein a significant load is detected at the instrument. In loading scenarios wherein loads are greater than zero but less than a maximum load, scaling would follow the plotted (312) configuration. FIG. 13B illustrates a plot (314) wherein a stepwise decrease in scaling factor changes the scaling factor to a next step down in ratio at each of a series of predetermined loading threshold points (316, 318, 320). In the event of a quick loading past the third threshold point (320), in this embodiment, scaling would be taken to zero, and moves at the master input device would not result in moves at the slave.

As described in the aforementioned incorporated by reference disclosures, a haptically-enabled master input device may be utilized to navigate the subject elongate instruments while providing the operator with mechanical feedback through the master input device. In one embodiment, haptic sensations may be delivered to the operator through the master input device which are indicative of the presence and/or quantity of loads applied to the distal portion of the instrument. In one embodiment, wherein a uniaxial load is detected, such as in certain variations of the aforementioned and incorporated Intellisense® technology, a vibration pattern may be delivered to the operator to indicate that a load is being applied, and amplitude and/or frequency of such vibration pattern may be varied in accordance with load quantity to provide the operator with indication of such quantity. For example, the following equations may be utilized to calculate a smooth sinusoidal force pattern in the presence of a shifting frequency:

Theta(t)=integral of (theta*2*pi*f(t)dt)

Theta[k]=theta[K−1]+theta*2*pi*f[k]Ts

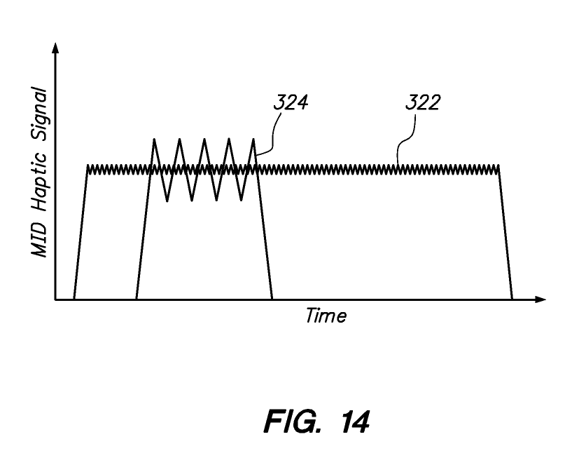

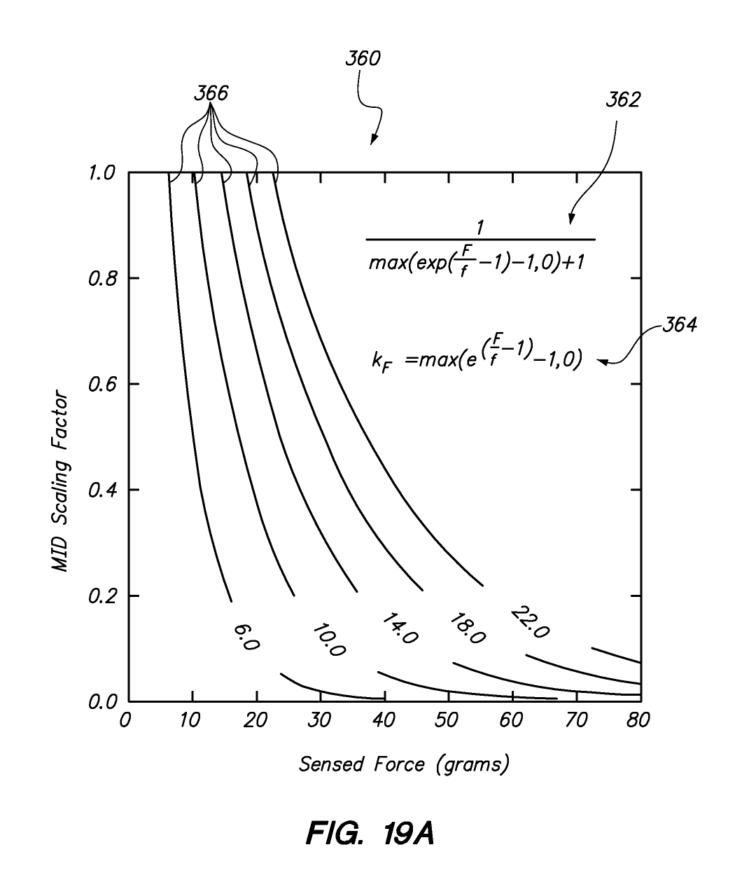

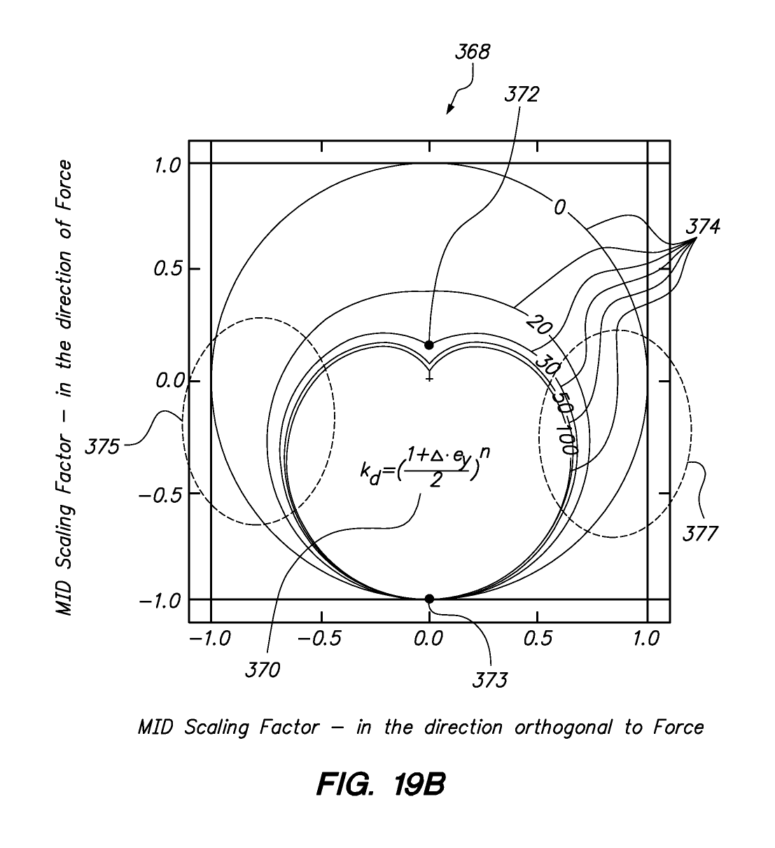

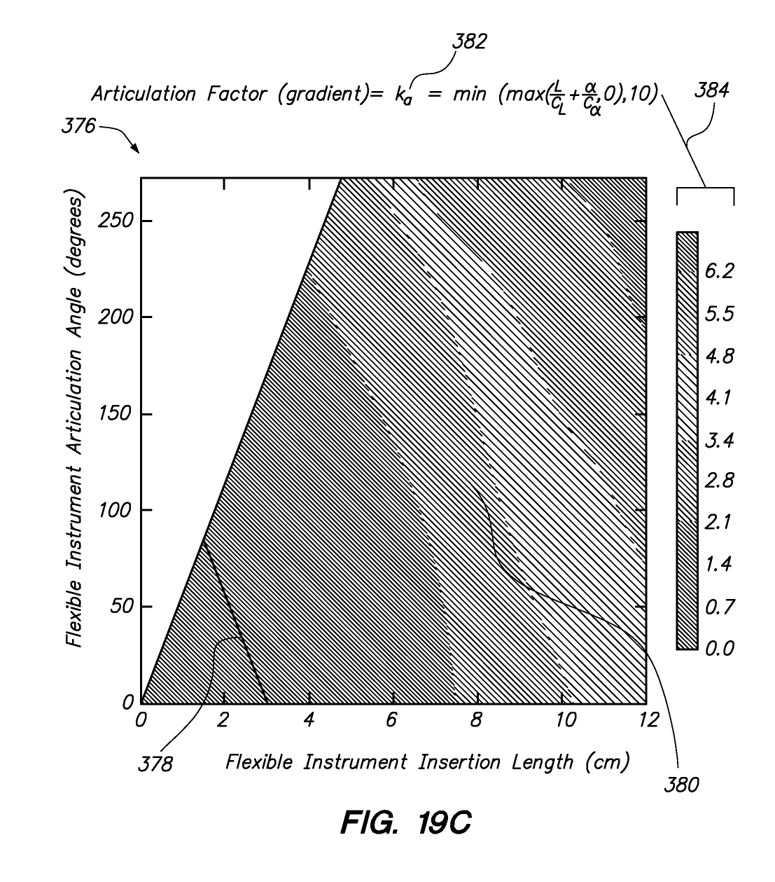

F[k]=A[k]*sin(theta[k])

Where f is the frequency, A is the desired amplitude, F is the force to be applied to the tool, Ts is the sample time, and theta is the phase through the current cycle. The frequency, amplitude, phase, and instantaneous force are all key attributes of the vibration object. In another embodiment, an additional vibratory pattern may be overlaid upon the first vibratory pattern, to indicate something else to the operator, such as current delivered through an instrument distal tip RF electrode, temperature sensed using one of the means described above, or other variables. Referring to FIG. 14, such an overlaying configuration is illustrated, with a higher frequency, lower amplitude plot (322) representing a vibratory pattern delivered to the operator of a haptic input device based upon a constant force applied at an instrument distal tip, for example, while an additional pattern (plot 324) may be also presented to the operator using the same master input device to provide an indication of some other treatment-related variable, such as sensed temperature, current delivery rate, power delivery, and the like, applied to tissues adjacent the distal instrument tip. Depending upon the quality and resolution of the haptic master input device, many variations of pluralities of vibratory feedback patterns may be imparted simultaneously to an operator of such a system to indicate the status of many states of variables such as load applied. For example, in one embodiment, a binary type of overlay signal may indicate merely the presence of a variable threshold crossing, such as a current density amount that is greater than a predetermined current density. In another embodiment, the overlay signal may not only indicate the existence of such variable, or variable threshold crossing, but also may be configured to scale with the quantification of such variable (i.e., greater current density, higher amplitude and/or frequency of the overlay signal). Other embodiments are described below in reference to FIGS. 19A-19C, wherein master input device motion scaling may be varied in relation to directionality of the instrument positioning, articulation of the instrument, insertion length of the instrument, and/or forces applied to or sensed by the instrument.

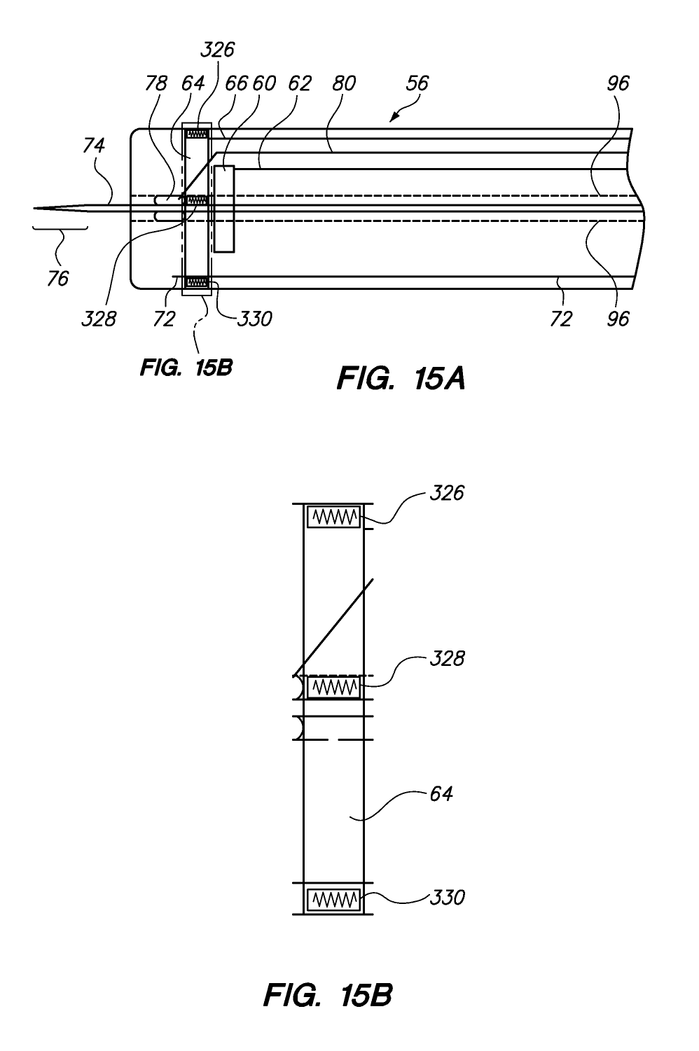

Referring to FIG. 15A, an instrument assembly (56) similar to that depicted in FIG. 3B is depicted, with the addition of three or more small discrete load sensors (326, 328, 330), such as resistive type strain gauges or other small load sensors, as described above. Such sensors (326, 328, 330) are shown in greater detail in the magnified view of FIG. 15B, and may be utilized to produce not only a reading of compressive or tensile forces applied to the distal tip of the instrument along the instrument's longitudinal axis, but also indications of force vectors for off axis loads applied, in three dimensions. Such three dimensional forces may be utilized in the determination and application of haptic feedback patterns and vectors thereof to the operator through a haptic master input device. Uniaxial force sensing, such as that featured in the aforementioned and incorporated Intellisense® technology, or three dimensional force sensing using an embodiment such as that described above in reference to FIGS. 15A and 15B, may be utilized clinically to provide contact patterns, lines, or drags with predetermined loading configurations. For example; in one embodiment, a curvilinear line pattern may be selected for an RF ablation drag within a chamber of the heart, and a constant axial force application prescribed for the contact pattern along the drag; alternatively, a predetermined force contour or profile (such as one wherein the force is decreased for the portion of the curvilinear treatment pattern that crosses a particularly load sensitive portion of substrate tissue structure).

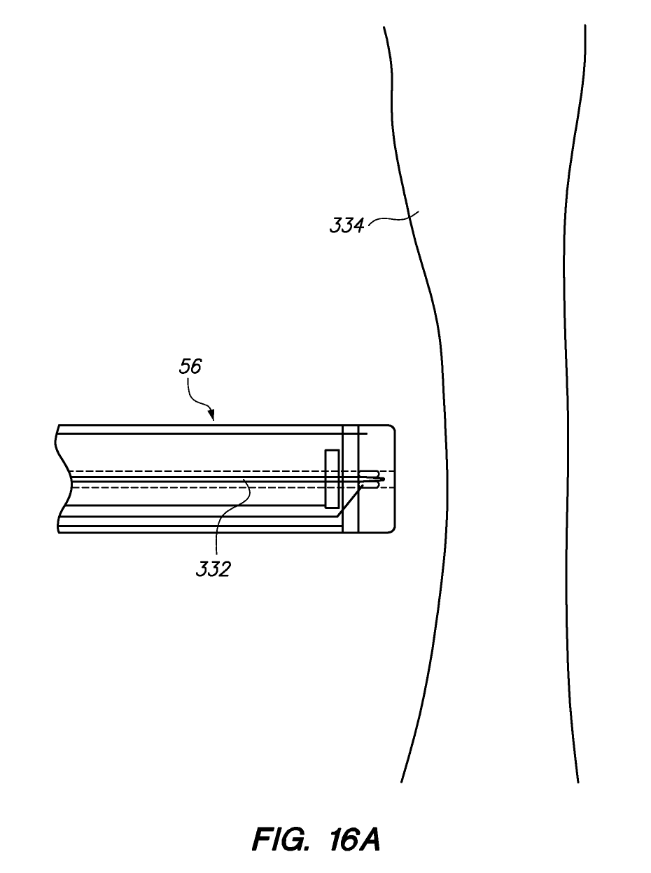

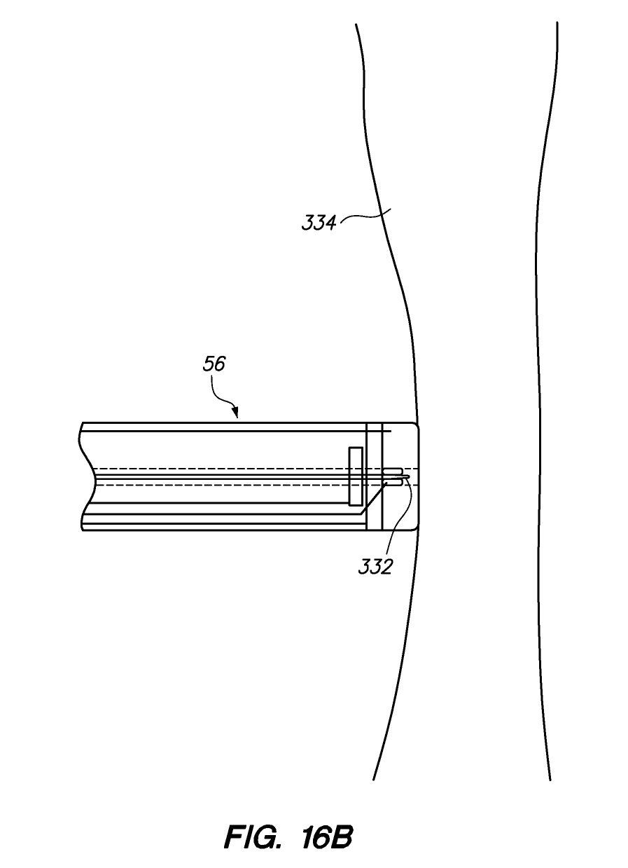

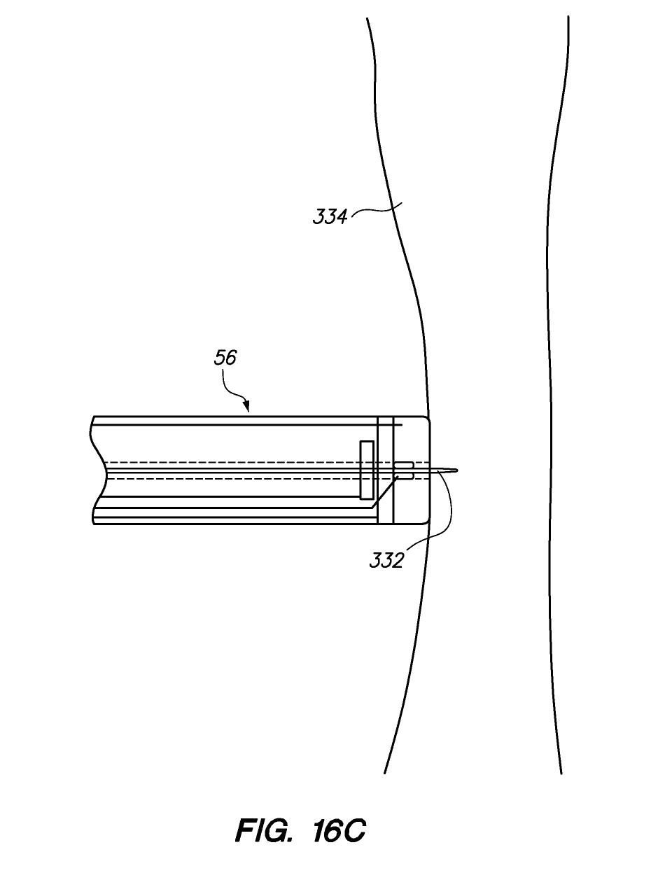

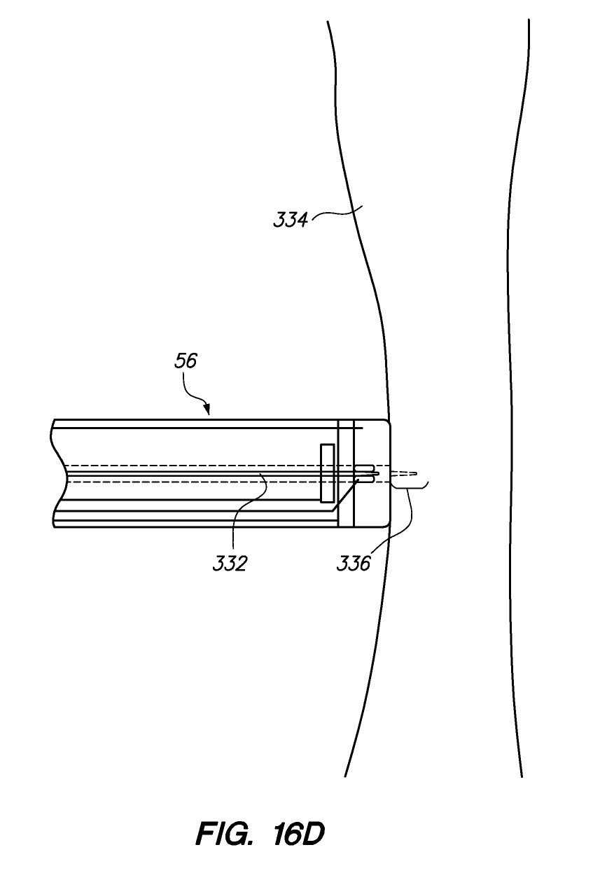

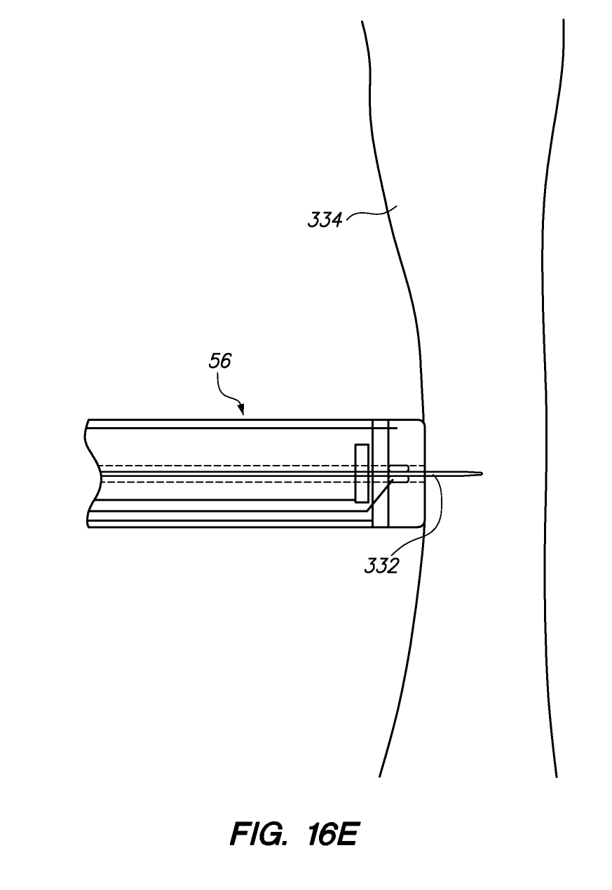

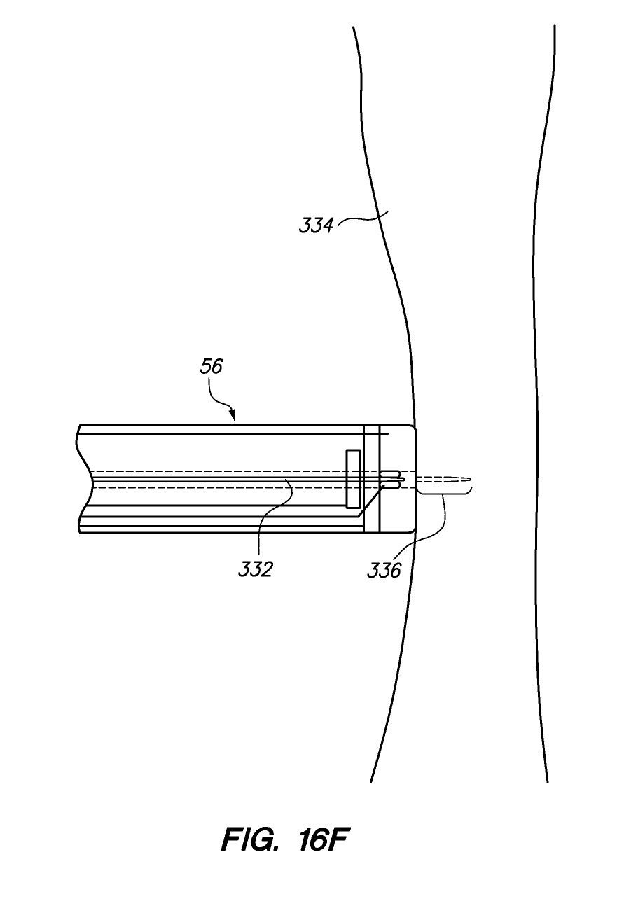

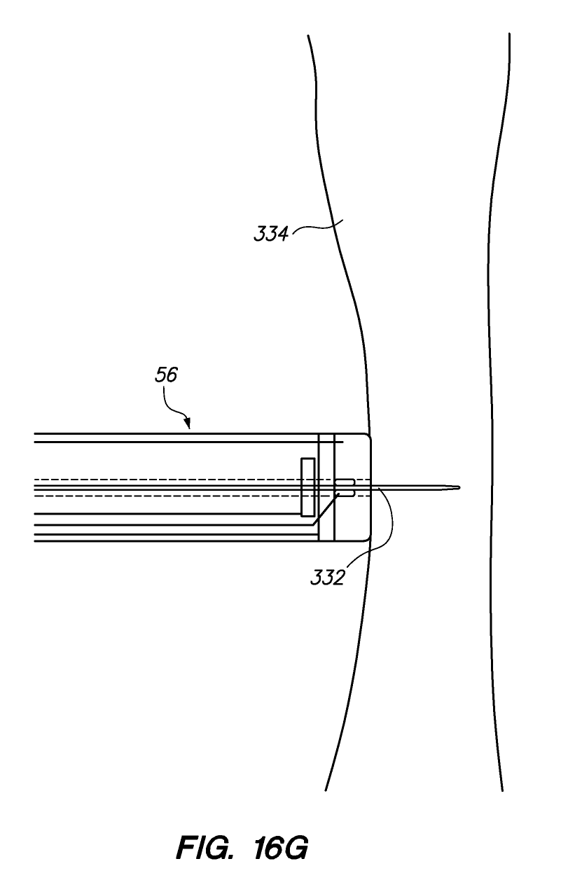

Referring to FIGS. 16A-16G, one embodiment of a procedure for removing material from an in situ interventional site is depicted. Referring to FIG. 16A, an instrument assembly similar to that depicted in FIG. 3B is depicted, having a drilling type of elongate probe (332) rather than a needle-like device as shown in FIG. 3B (element 74 of FIG. 3B). The assembly is depicted approaching a calcified tissue structure (334), such as a portion of the human spine. Referring to FIG. 16B, the instrument assembly is shown immediately adjacent the calcified tissue structure (334) where sensors comprising the instrument assembly may be utilized to detect information regarding the immediate portions of such tissue structure, such as compliance to applied low levels of axial loading, conductivity, or temperature. Referring to FIG. 16C, the drilling member (332) may be advanced into the calcified tissue structure (334), and later withdrawn, as shown in FIG. 16D, leaving behind a defect (336). Referring to FIG. 16D, the drilling instrument (332) may be advanced yet further, creating an opportunity to use sensing techniques, such as tissue compliance sensing, to analyze the scenario clinically from another deeper perspective. FIG. 16F shows another cycle of withdrawal, and FIG. 16G shows another cycle of insertion and further advancement. Such cyclic insertion and withdrawal, along with sensing during such intervention, may be highly advantageous in the case of a tissue removal intervention, such as one wherein cancerous or necrotic tissue is to be removed, and healthy substrate tissue left in place. Given a difference between the desirably removed tissue and the tissue to be left in place, that may be sensed with the instrument system, such procedures may be streamlined. For example, it may be known that necrosed bone material has a different conductivity, temperature, and/or mechanical compliance. In such a scenario, load sensing, temperature sensing, and or conductivity sensing at the distal tip of the instrument assembly may be used as tissue is incrementally removed. In other words, the instrument may be advanced, an incremental amount of material removed, and compliance (or whatever other variable may be sensed, analyzed, and correlated to a known tissue state) tested; if the tested compliance is greater than a threshold that is correlated with non-necrosed bone, another cycle of advancement, removal, and analysis is conducted—until less compliant bone, correlated with healthy bone, is reached, after which the advancement of the instrument may be ceased. Further, once the advancement has been ceased, the robotic instrument control system may be utilized to determined with reasonable precision the volume of the defect created, which may be useful for subsequent defect filling with materials such as poly methyl methacrylate or the like.

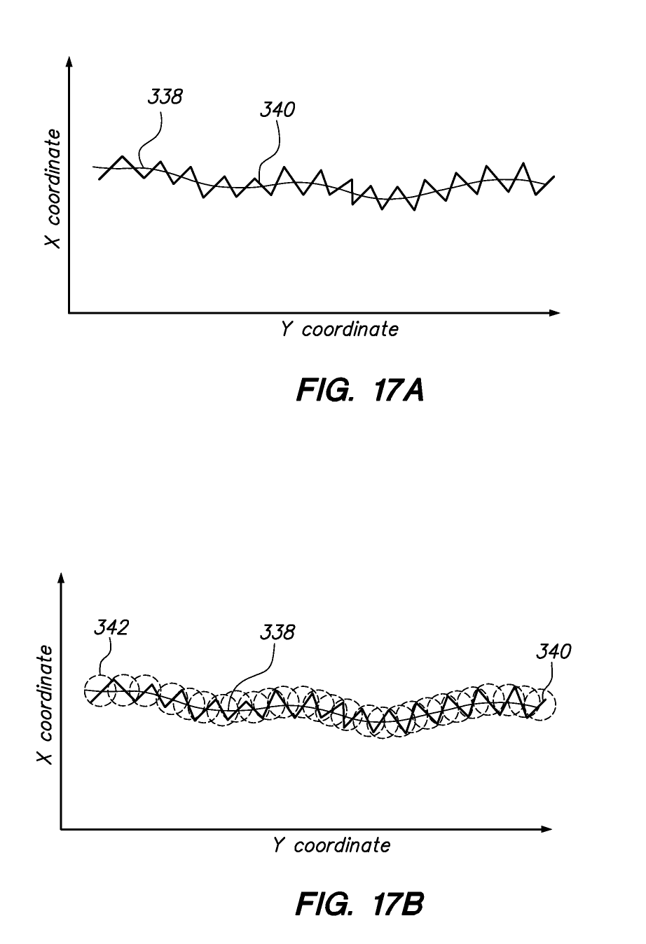

Referring to FIG. 17A, when a fairly linear or curvilinear treatment pathway (338), such as a long linear lesion ablation “burn”, has been selected, a zig zagging type of interventional pattern (340) may improve the knowledge of the anatomy, physiology, and treatment by allowing an instrument assembly comprising sensors, such as those depicted in FIG. 3A-4E, or 15A-B, to gather more data regarding the region and treatment. In other words, if the instrument strictly follows the curvilinear pathway (340) during both treatment periods and non-treatment navigation periods, it is sampling data only from that area—whereas if it intentionally navigates a bit farther afield between treatments, it gathers more data to facilitate a more refined understanding of the clinical scenario. One advantage of an electromechanically controlled instrument is that such zig sagging, or other pattern, may be automated. For example, referring to FIG. 17B, the zig sagging pattern of movement (340) may allow the distal tip of the instrument to encounter, and sense with pertinent sensor capabilities, three or more times the tissue swath, depending upon the amplitude of the zig sagging pattern (340), while also creating a curvilinear lesion sufficient to block aberrant conduction pathways from crossing the predetermined curvilinear path (338), the curvilinear lesion comprising an aggregation of smaller lesions (342) created, for example, at the intersections of the zig sagging pattern (340) with the predetermined curvilinear pattern (338). The widened swath essentially provides a larger sample size for pertinent analysis of the situation.

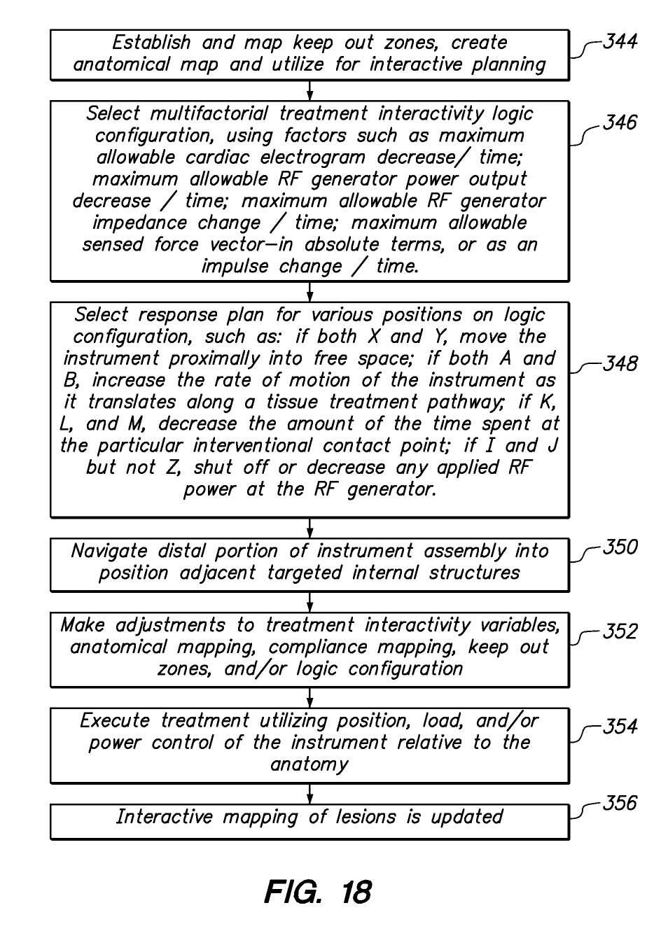

Referring to FIG. 18, an embodiment is depicted to illustrate that multifactorial analysis may be conducted with treatments in situ, depending upon predetermined, and interactively adjustable, variable or factor interactivity logic. For example, after establishing keep out zones, creating an anatomical map, and generally creating an interventional plan to control tissue/instrument physical interaction (344), multifactorial logic may be configured (346) to utilize a plurality of sensed factors, such as those described in reference to FIG. 12 (300). A response plan (348) may also be selected or created, to control the interactivity of sensed factors and interventional variables. For example, one variable may be deemed controlling in certain situations, while another may become dominant from a controls perspective in another, such as in a scenario wherein if a sensed temperature is too high and a sensed force is too high, the instrument is to be pulled proximally into free space—but not if only one of these factors is higher than a predetermined threshold. Many combinations of variables may be coded into the logic and response plans. Subsequently, these configurations may be employed as the instrument assembly is navigated (350), and adjustments may be made (352) while treatment is executed (354) and interactive mapping is updated (356). For example, in one multivariate treatment embodiment, distal temperature, nearby tissue structure compliance, distal instrument load, and current delivery density per unit area of tissue structure may be simultaneously monitored, and the logic may be configured to stop application of treatment energy when a temperature, load, or current delivery density is exceeded, but not if a compliance threshold is exceed, unless the compliance threshold is crossed along with a significant decrease in detected distal load.

Instrument motion may be a scaled version of master device commanded motion based on a variety of other factors, e.g. forces, configurations and/or motion directions. Where force is measured, one embodiment would emulate a pre-determined motion-force relationship with the master. Alternatively, a more heuristic approach may be implemented. Referring to FIGS. 19A-19C, in one embodiment, motion scaling at the master input device may be varied in accordance with the following relationship:

Xcatheter=kₜx_{MiD}

wherein Xcatheter represents commanded instrument (in one embodiment a steerable catheter) motion utilized by the system to move the instrument, Xmid represents motion commanded at the master input device (“MID”) by the operator, and Kt represents a total scaling factor comprised of three components, per the equation below in one embodiment, including a force component Kf, an instrument direction component Kd, and an instrument articulation/insertion component Ka:

$${k}_{t} = \frac{{k}_{a} {k}_{d}}{( 1 + {k}_{F} )} + 1 - {k}_{d} .$$

Referring to FIG. 19A, MID motion scaling factor is plotted (360) versus sensed insertion axis force (measured, for example, using the Intellisense® technology described above) for one implementation of a Kf relationship (364, 362) wherein motion scaling is generally decreased as sensed force magnitude is increased for various quantitative levels of Kf (366); portions of the depicted relationships are linear, while others are nonlinear. In other words, when a relatively high insertion (i.e., compressive) force is detected, motion scaling at the MID is generally decreased—to effectively “gear down” the MID-operator control relationship. In the depicted equation (364, 362), “F” represents the measured force, and “f” represents the force scaling factor listed on each of the plots (366).

Referring to FIG. 19B, MID scaling factor is plotted (368) versus the directionality of the force applied for one implementation embodiment. For example, in the depicted embodiment, with a sensed load equal to 30 grams (plots 374 are shown for sensed loads of 0 grams, 20 grams, 30 grams, 50 grams, and 100 grams), straight outward insertion (i.e., compressive along the load sensing axis—see point 372) is scaled at approximately 0.2 (or twenty percent ratio of manually input command motion to output command motion to the system; geared down quite significantly), while straight withdrawal of the instrument (i.e., along the load sensing axis—see point 373) is scaled at 1.0 (i.e., a 1:1 ratio of manually input command motion to output command motion to the system; effectively no scaling; the theory being that withdrawal generally is safe and should be able to be expediently directed by the MID). Motion in lateral directions orthogonal to the load sensing axis (for example, if the load sensing axis is “Z”, lateral motion would be in the “X” and/or “Y” directions) may be scaled with a smooth connecting relationship (see plotted regions 375 and 377 in the exemplary embodiment) configured to avoid disjointed motion or any jumping or unpredictable instrument motion relative to commands at the MID. With a zero sensed load, scaling in the depicted embodiment is set at 1.0 in all directions; as load is increased, the most sensitivity (and most downscaling at the MID) in the depicted embodiment is for insertion type movements that are generally against the applied load (i.e., like to increase the sensed load). In the depicted Kd equation (370), the delta symbol represents the normalized direction of MID motion (a vector in R³), and e_{y} is the unit vector in the direction of the instrument tip. Higher values of “n” will tighten the directionality of the scaling forward (i.e., lateral motion will be less scaled for higher values of “n”).

Referring to FIG. 19C, an articulation/insertion (“Ka”) factor (382) embodiment is plotted (376) to show that a gradient (380) may be implemented wherein motion scaling (384) is highest when the instrument bending articulation angle is lowest, instrument insertion length (i.e., the amount of elongate instrument body that is inserted past structural support provided by other instrument-related structures such as introducer sheaths) is the lowest, or both. From a mechanics of materials perspective, the composite instrument generally is at its stiffest when it is maximally withdrawn and not bent (i.e., straight)—and this is when, in the depicted embodiment, motion at the MID is scaled down the most. When the instrument is maximally inserted, or when the instrument is maximally articulated (i.e., bent, in the scenario of a remotely controllable steerable catheter) the instrument is more highly compliant or flexible in relation to applied loads—and this is when, in the depicted embodiment, the motion scaling is minimized. Such configuration may be deemed a “virtual compliance” scaling modality, wherein the scaling is configured to have the instrument make only small incremental “soft touch” motions when the instrument is in a naturally stiffer configuration, and to be more quickly movable with less scaling when the instrument is in a configuration wherein it is naturally more akin to “soft touch”—thus providing the operator with a spectrum of “soft touch” operation. In the depicted equation (382), “L” represents the length of instrument insertion in centimeters, alpha is the instrument tip articulation in radians, and C_{L} and Cₐₗₚₕₐ are insertion and articulation factors, respectively. The plot (380) depicts the sample implementation wherein both of these factors are equal to 3.0, and the line (378) depicts unity scaling due to articulation (i.e., the scaling factor is neither increased nor decreased by the articulation or bending component).

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. A robotic catheter system, comprising:
a. a controller including a master input device; and
b. an electromechanically steerable elongate instrument having a proximal interface portion and a distal portion, the proximal interface portion being configured to be operatively coupled to an electromechanical instrument driver in communication with the controller, the distal portion being configured to be interactively navigated adjacent internal tissue structures of a patient's body in response to signals from the controller;
wherein the controller is operatively coupled to a treatment interactivity variable sensor selected from the group consisting of: a cardiac electrogram electrode, an RF generator power output sensor, an instrument distal portion impedance sensor, and an instrument distal portion force sensor; and
wherein the controller is configured to affect the operation of the electromechanically steerable elongate catheter by automatically executing an instrument movement or treatment command based upon changes in information received from said treatment interactivity variable sensor.

2. The robotic catheter system of claim 1, wherein the controller is operatively coupled to a cardiac electrogram electrode, and wherein upon sensing that the amplitude of a cardiac electrogram signal from the cardiac electrogram electrode has changed over time at a rate exceeding a predetermined threshold rate, the controller is configured to move the instrument to another position or increase the rate of movement of the instrument.

3. The robotic catheter system of claim 1, wherein the controller is operatively coupled to a cardiac electrogram electrode and an RF generator configured to transmit energy to the distal portion of the elongate instrument at a transmission rate to treat tissues adjacent thereto, and wherein upon sensing that the amplitude of a cardiac electrogram signal from the cardiac electrogram electrode has changed over time at a rate exceeding a predetermined threshold rate, the controller is configured to change the transmission rate.

4. The robotic catheter system of claim 1, wherein the controller is operatively coupled to an RF generator configured to transmit energy to an electrode positioned at the distal portion of the elongate instrument at a transmission rate to treat tissues adjacent thereto, and wherein upon sensing that the transmission rate has changed over time at a rate exceeding a predetermined threshold rate, the controller is configured to move the instrument to another position or increase the rate of movement of the instrument.

5. The robotic catheter system of claim 1, wherein the controller is operatively coupled to an RF generator configured to transmit energy to an electrode positioned at the distal portion of the elongate instrument at a transmission rate to treat tissues adjacent thereto, and wherein upon sensing that the transmission rate has changed over time at a rate exceeding a predetermined threshold rate, the controller is configured to change the transmission rate.

6. The robotic catheter system of claim 1, wherein the controller is operatively coupled to an impedance sensor coupled to the distal portion of the elongate instrument, and wherein upon sensing that the impedance has changed over time at a rate exceeding a predetermined threshold rate, the controller is configured to move the instrument to another position or increase the rate of movement of the instrument.

7. The robotic catheter system of claim 1, wherein the controller is operatively coupled to an impedance sensor coupled to the distal portion of the elongate instrument and an RF generator configured to transmit energy to the distal portion of the elongate instrument at a transmission rate to treat tissues adjacent thereto, and wherein upon sensing that the impedance has changed over time at a rate exceeding a predetermined threshold rate, the controller is configured to change the transmission rate.

8. The robotic catheter system of claim 1, wherein the controller is operatively coupled to force sensor configured to sense interfacial forces between the distal portion of the elongate instrument and adjacent structures, and wherein upon sensing that a sensed force applied at a sensed vector has changed over time at a rate exceeding a predetermined threshold rate or exceeds a predetermined threshold value, the controller is configured to move the instrument to another position in a direction opposite to the sensed force vector.

9. The robotic catheter system of claim 8, wherein the controller is configured to continue moving the instrument in the direction opposite to the sensed forced vector until the sensed force vector decreases by a predetermined percentage, or to a preset threshold value.

10. A method for operating a robotic catheter system, comprising:
transmitting a movement command generated with a master input device to a controller, the controller being operatively coupled to an electromechanically steerable elongate instrument having a proximal interface portion and a distal portion, the proximal interface portion being configured to be operatively coupled to an electromechanical instrument driver in communication with the controller, the distal portion being configured to be interactively navigated adjacent internal tissue structures of a patient's body in response to signals from the controller;
wherein the controller is operatively coupled to a treatment interactivity variable sensor selected from the group consisting of: a cardiac electrogram electrode, an RF generator power output sensor, an instrument distal portion impedance sensor, and an instrument distal portion force sensor; and
wherein the controller is configured to affect the operation of the electromechanically steerable elongate catheter by automatically executing an instrument movement or treatment command based upon changes in information received from said treatment interactivity variable sensor.

11. The method of claim 10, wherein the controller is operatively coupled to a cardiac electrogram electrode, and wherein upon sensing that the amplitude of a cardiac electrogram signal from the cardiac electrogram electrode has changed over time at a rate exceeding a predetermined threshold rate, the controller is configured to move the instrument to another position or increase the rate of movement of the instrument.

12. The method of claim 10, wherein the controller is operatively coupled to a cardiac electrogram electrode and an RF generator configured to transmit energy to the distal portion of the elongate instrument at a transmission rate to treat tissues adjacent thereto, and wherein upon sensing that the amplitude of a cardiac electrogram signal from the cardiac electrogram electrode has changed over time at a rate exceeding a predetermined threshold rate, the controller is configured to change the transmission rate.

13. The method of claim 10, wherein the controller is operatively coupled to an RF generator configured to transmit energy to an electrode positioned at the distal portion of the elongate instrument at a transmission rate to treat tissues adjacent thereto, and wherein upon sensing that the transmission rate has changed over time at a rate exceeding a predetermined threshold rate, the controller is configured to move the instrument to another position or increase the rate of movement of the instrument.

14. The method of claim 10, wherein the controller is operatively coupled to an RF generator configured to transmit energy to an electrode positioned at the distal portion of the elongate instrument at a transmission rate to treat tissues adjacent thereto, and wherein upon sensing that the transmission rate has changed over time at a rate exceeding a predetermined threshold rate, the controller is configured to change the transmission rate.

15. The method of claim 10, wherein the controller is operatively coupled to an impedance sensor coupled to the distal portion of the elongate instrument, and wherein upon sensing that the impedance has changed over time at a rate exceeding a predetermined threshold rate, the controller is configured to move the instrument to another position or increase the rate of movement of the instrument.

16. The method of claim 10, wherein the controller is operatively coupled to an impedance sensor coupled to the distal portion of the elongate instrument and an RF generator configured to transmit energy to the distal portion of the elongate instrument at a transmission rate to treat tissues adjacent thereto, and wherein upon sensing that the impedance has changed over time at a rate exceeding a predetermined threshold rate, the controller is configured to change the transmission rate.

17. The method of claim 10, wherein the controller is operatively coupled to force sensor configured to sense interfacial forces between the distal portion of the elongate instrument and adjacent structures, and wherein upon sensing that a sensed force applied at a sensed vector has changed over time at a rate exceeding a predetermined threshold rate or exceeds a predetermined threshold value, the controller is configured to move the instrument to another position in a direction opposite to the sensed force vector.

18. The method of claim 17, wherein the controller is configured to continue moving the instrument in the direction opposite to the sensed forced vector until the sensed force vector decreases by a predetermined percentage, or to a preset threshold value.
